(19) United States
(12) Patent Application Publication
(10) Pub. No.: US 2025/0352494 A1
(43) Pub. Date: Nov. 20, 2025

(54) TRANSDERMAL DELIVERY DEVICE FOR PEPTIDE DELIVERY AND METHODS OF USE

(71) Applicants: PassPort Technologies, Inc., San Diego, CA (US); National University Corporation Hokkaido University, Sapporo-Shi, Kita-ku, Hokkaido (JP); National University Corporation Hamamatsu University School of Medicine, Higashi-Ku Hamamatsu-Shi, Shizuoka (JP)

(72) Inventors: Hirotoshi Adachi, San Diego, CA (US); Jeremiah Meloch, San Diego, CA (US); Melissa Lerman, San Diego, CA (US); Yuki Yamada, Tokushima (JP); Toshiharu Suzuki, Sapporo (JP); Haruka Yasuda, Ibaraki (JP); Yasuomi Ouchi, Hamamatsu (JP)

(21) Appl. No.: 18/872,774

(22) PCT Filed: Jun. 8, 2023
(86) PCT No.: PCT/US2023/068150

§ 371 Date: Dec. 6, 2024

Related U.S. Application Data
(60) Provisional application No. 63/350,982, filed on Jun. 10, 2022.

(51) Int. Cl. A61K 9/70; A61K 38/17

(52) U.S. Cl. CPC A61K 9/7084; A61K 38/1709

(57) ABSTRACT

Disclosed herein are patches, methods, devices, and systems for delivering a non-aggregating peptide, such as alcadein and its fragments, into a subject. In some aspects, the patch includes a backing, a matrix comprising a non-aggregating peptides disposed within the matrix, and a release liner. In other aspects, the method includes opening at least one channel in the subject's skin, applying the patch described herein, thereby treating a disease or disorder associated with the brain, such as Alzheimer's disease.

### CROSS-REFERENCE TO RELATED APPLICATIONS

This application claims the benefit of U.S. Provisional Application No. 63/350,982 filed on Jun. 10, 2022, which is incorporated by reference in its entirety.

### REFERENCE TO SEQUENCE LISTING

The present application is filed with a Sequence Listing in Electronic format. The Sequence Listing is provided as a file entitled PPORT.008WO_ST_26.xml, created Jun. 6, 2023, which is approximately 7 kb in size. The information in the electronic format of the sequence listing is incorporated herein by reference in its entirety.

### BACKGROUND

### Field

This application relates to compositions, devices and methods for transdermal drug delivery, and in particular to peptide compositions and methods for administering the peptide to subjects by transdermal microporation devices.

### Description

Alzheimer's disease (Aβ) is the most common senile dementia disease. The increase of AD patients is a serious problem in the world. Familial gene mutations that increase the risk of developing AD have been found, but most of the cases are thought to be cognitive decline due to age-related changes in the brain. Therefore, although aging is the greatest risk factor for developing AD, a characteristic change commonly seen in AD patients is the accumulation of amyloid β (Aβ) in the brain. Aβ, which increases with age, forms aggregates of Aβ oligomer in the brain and develops cytotoxicity. Since the decline in cognitive function caused by Aβ oligomer has been reported, Aβ has been considered to be a strong cause of AD onset.

The nerve-specific membrane protein Alcadein β is metabolized by a pathway similar to that of Aβ to produce p3-Alcβ. Unlike Aβ, which accumulates in the brain exponentially with aging, p3-Alcβ does not accumulate in the brain and decreases from the cerebrospinal fluid CSF) with aging, and the p3-Alcβ in CSF further decreases significantly in AD patients compared to age-matched non-demented-subjects, so it has been thought to be involved in AD onset. In addition, p3-Alcβ does not have aggregation, unlike Aβ, alleviates neuronal toxicity caused by Aβo, and cognitive function by peripheral administration in Aβ oligomer-induced acute cognitive function model mice. It has been clarified that it shows a reduction and improvement effect. Therefore, an increase in toxic Aβ oligomer and a decrease in p3-Alcβ, which has an inhibitory effect, are considered to be involved in the onset and progression of AD. Since the action of p3-Alcβ is carried out by its partial peptide p3-Alcβ9-19, by allowing p3-Alcβ9-19 to reach the brain, the onset and progression of AD associated with the decrease of the endogenous molecule p3-Alcβ can be prevented.

Various peptide compositions and methods of treatment have been suggested for AD. However, peptide drugs are generally unstable in the living body and difficult to deliver to the brain. Thus, there remains a long-felt need for improved peptides, formulations, compositions, devices and methods for the delivery of peptides to pass through the blood brain barrier.

### SUMMARY OF THE DISCLOSURE

Some aspects of the disclosure relate to a patch for delivering a non-aggregating peptide into a subject. In some embodiments, the patch includes a backing, a matrix including a non-aggregating peptide disposed within the matrix, and a release liner, wherein the release liner is configured to be removed before application to the subject's skin. In some embodiments, the non-aggregating peptide is p3-Alcβ. In some embodiments, the p3-Alcβ is selected from at least one of p3-Alcβ1-40, p3-Alcβ1-37, p3-Alcβ9-19, p3-Alcβ1-19, p3-Alcβ11-19 or its derivatives. In some embodiments, the p3-Alcβ is p3-Alcβ9-19 or its derivatives. In some embodiments, the non-aggregating peptide is in an amount in the range of about 0.01 mg/cm² and 200 mg/cm². In some embodiments, the matrix further includes at least one sugar. In some embodiments, the at least one sugar is selected from a non-reducing sugar, a reducing sugar, or a combination thereof. In some embodiments, the non-reducing sugar is selected from sucrose, trehalose, mannitol, sorbitol, or a combination thereof. In some embodiments, the reducing sugar is selected from lactose, maltose, or a combination thereof. In some embodiments, the ratio of the at least one sugar to non-aggregating peptide is greater than 0.02. In some embodiments, the ratio of the at least one sugar to non-aggregating peptide is from about 0.02 to about 0.4. In some embodiments, the matrix further includes an organic acid, organic salt, or a combination of thereof. In some embodiments, the organic acid is a Pharmacokinetic (PK) modifier. In some embodiments, the PK modifier is citric acid and its salt form. In some embodiments, the matrix further includes a preservative. In some embodiments, the preservative is an anti-microbial agent. In some embodiments, in the anti-microbial agent is selected from methylparaben, propylparaben, benzalkonium chloride, and sodium benzoate. In some embodiments, the matrix includes at least one fiber. In some embodiments, the at least one fiber is a non-woven fiber. In some embodiments, the at least one fiber has a thickness of less than 300 μm. In some embodiments, the at least one fiber has a weight of less than 100 g/m². In some embodiments, the matrix has a water-holding capacity that is less than 10 mg/cm². In some embodiments, the matrix includes a laminated material of film and at least one fiber. In some embodiments, the matrix further includes at least one of sucrose, lactose, disodium citrate sesquihydrate, methylparaben, propylparaben, and benzalkonium chloride.

Some aspects relate to methods for treating a disease or condition associated with the brain in a subject. In some embodiments, the method includes opening at least one channel in the subject's skin and applying the patch as described herein to the subject's skin, thereby treating the disease or condition associated with the brain. In some embodiments, the disease or condition associated with the brain is a neurodegenerative disease. In some embodiments, the neurodegenerative disease is Alzheimer's disease. In some embodiments, opening at least one channel in the subject's skin includes applying a transdermal microporation device to the subject's skin. In some embodiments, the transdermal microporation device utilizes a thermal tissue ablation by using a filament array having a plurality of filaments that are disposed in the skin of the subject, wherein each filament is capable of conductively delivering thermal energy via direct contact to the tissue membrane to form the plurality of micropores in a micropore area of the tissue membrane. In some embodiments, the transdermal microporation apparatus creates between about 25 to about 500 micropathways/cm². In some embodiments, the transdermal microporation apparatus has a poration energy from about 2 to about 6 mJ/filament. In some embodiments, opening at least one channel in the subject's skin has an area from about 0.25 cm² to about 4 cm². In some embodiments, the transdermal microporation apparatus is a microneedle, laser, or radio frequency device capable of producing one or more micropores in the subject's skin. In some embodiments, applying the patch increases neuronal viability in the subject. In some embodiments, applying the patch increases mitochondrial activity in the brain of the subject. In some embodiments, the patch provides a maximum blood concentration of the non-aggregating peptide at least 0.5 hour after administration. In some embodiments, the non-aggregating peptide is maintained for at least 6 hours after administration of the patch. In some embodiments, the non-aggregating peptide from the patch is maintained in the blood of the subject for at least 6 hours after administration of the patch to the subject. In some embodiments, the transfer rate of the non-aggregating peptide from the blood of the subject to the cerebrospinal fluid of the subject is at least 2%.

Some aspects relate to a transdermal delivery system for delivering a non-aggregating peptide into a subject. In some embodiments, the system includes a patch as described herein and a transdermal microporation device. In some embodiments, the transdermal microporation device includes an applicator electrically connected to an array of conductive filaments, wherein the transdermal microporation device is configured to generate thermal energy based on a current flowing through the array of conductive filaments, and provide the thermal energy to a biological membrane positioned adjacent to the transdermal microporation device, and a power supply circuit configured to provide the current to the transdermal microporation device. In some embodiments, the applicator supplies a predetermined electrical energy to the array of conductive filaments thereby creating one or more micropores. In some embodiments, the patch is applied to the one or more micropores. In some embodiments, the transdermal microporation device creates between about 25 to about 500 micropathways/cm². In some embodiments, the transdermal microporation device has a poration energy from about 2 to about 6 mJ/filament. In some embodiments, opening at least one channel in the subject's skin has an area from about 0.25 cm² to about 4 cm². In some embodiments, the one or more micropores is about 0.5 to about 12.5% of the total poration area. In some embodiments, the transdermal microporation device is a microneedle, laser, or radio frequency device capable of producing one or more micropores in the subject's skin. In some embodiments, the transdermal microporation device produces at least 50 pores in a subject's skin. In some embodiments, the patch further includes a drug pellet.

### BRIEF DESCRIPTION OF THE DRAWINGS

The accompanying drawings, which are incorporated in and constitute a part of this specification, illustrate several aspects described below and together with the description, serve to explain the principles of the disclosure. Like numbers represent the same elements throughout the figures.

FIG. 1 illustrates amino acid sequences of Alcβ and p3-Alcβ.

FIG. 2A illustrates an example of a patch. FIG. 2B illustrates an application of a patch containing a peptide drug with transdermal microporation. After microporation to skin, micropathways are created in Stratum Corneum and Epidermis layers. The dry patch containing a peptide drug in matrix is applied on the microporated area. The dissolved drug migrates to the body via micropathways.

FIG. 3A illustrates a line graph depicting PK profiles of p3-Alcβ9-19 after intravenous administration in rats; FIG. 3B illustrates a line graph depicting PK profiles of p3-Alcβ9-19 after subcutaneous administration; FIG. 3C illustrates a logarithmic scale graph depicting PK profiles of p3-Alcβ9-19 in logarithmic scale.

FIG. 4A illustrates a line graph depicting dose responses of p3-Alcβ9-19 after intravenous administration in rates; FIG. 4B illustrates a line graph depicting dose responses of p3-Alcβ9-19 after subcutaneous administration in rats.

FIG. 5A illustrates a line graph depicting IR formulations; FIG. 5B illustrates a line graph depicting dose response from transdermal microporation.

FIG. 6A illustrates a logarithmic scale graph depicting changes in blood and cerebrospinal fluid concentration of p3-Alcβ9-19 after application of IR formulation containing p3-Alcβ9-19 with transdermal microporation in mice (400 density, 3 mJ/filament). FIG. 6B illustrates a logarithmic scale graph depicting changes in blood and cerebrospinal fluid concentration of p3-Alcβ9-19 after application of SR formulation containing p3-Alcβ9-19 with after transdermal microporation in mice (100 density, 3 mJ/filament).

FIG. 7 illustrates two images depicting changes in mitochondrial activity in the brain after p3-Alcβ9-19 transdermal microporation in monkeys.

FIG. 8 illustrates two bar graphs describing the intensities in mitochondrial activity in various parts of the brain after p3-Alcβ9-19 transdermal microporation (data analyzed from FIG. 6).

FIG. 9 illustrates a bar graph depicting the percentage increase in mitochondrial activity in various parts of the brain after p3-Alcβ9-19 transdermal microporation (equivalent to 0.5 and 1.0 mg/kg dose of p3-Alcβ9-19).

FIG. 10A illustrates a line graph depicting the PK profiles using non-reducing sugar formulations and p3-Alcβ concentration over time; FIG. 10B illustrates a logarithmic scale graph depicting the p3-Alcβ concentration over time.

FIG. 11 illustrates a line graph depicting non-reducing and reducing sugars enhanced p3-Alcβ absorption.

FIG. 12A illustrates a line graph depicting microporation conditions to control PK profiles using a reducing sugar with or without a PK modifier; FIG. 12B illustrates a logarithmic scale graph depicting microporation conditions to control PK profiles using a reducing sugar with or without a PK modifier.

FIG. 13 illustrates a bar graph depicting effects of microporation condition on p3-Alcβ9-19 delivery.

FIG. 14 illustrates the effects of enhancer content (reducing sugar) in p3-Alcβ transdermal microporation.

FIG. 15 illustrates a line graph comparing the AUC versus lactose content.

FIG. 16 illustrates a line graph depicting an AUC versus dose profile on IR formulations.

FIG. 17 illustrates a line graph depicting an AUC versus dose profile on SR formulations.

FIG. 18 illustrates a line graph depicting PK profiles of an optimized transdermal microporation formulation for immediate delivery.

FIG. 19A illustrates a line graph depicting a dose-dependency on p3-Alcβ9-19 patch in rats (AUC); FIG. 19B illustrates a line graph depicting a dose-dependency on p3-Alcβ9-19 patch in rats (Cmax).

### DETAILED DESCRIPTION

The present disclosure can be understood more readily by reference to the following detailed description, examples, drawing, and claims, and their previous and following descriptions. However, before the present devices, systems, and/or methods are disclosed and described, it is to be understood that this disclosure is not limited to the specific devices, systems, and/or methods disclosed unless otherwise specified. It is also to be understood that the terminology used herein is for the purpose of describing particular aspects only and is not necessarily intended to be limiting.

This description is provided as an enabling teaching of the disclosure. To this end, those skilled in the relevant art will recognize and appreciate that many changes can be made to the various aspects of the disclosure described herein, while still obtaining beneficial results. It will also be apparent that some of the desired benefits can be obtained by selecting some of the features described herein without utilizing other features. Accordingly, those who work in the art will recognize that many modifications and adaptations to the present description are possible and can even be desirable in certain circumstances and are a part of the present disclosure. Thus, this description is provided as illustrative of certain principles of the present disclosure and not in limitation thereof.

### Definitions

Unless defined otherwise, all technical and scientific terms used herein have the same meaning as is commonly understood by one of ordinary skill in the art. All patents, applications, published applications, and other publications referenced herein are incorporated by reference in their entirety unless stated otherwise. In the event that there are a plurality of definitions for a term herein, those in this section prevail unless stated otherwise.

As used throughout, the singular forms “a,” “an,” and “the” include plural referents unless the context clearly dictates otherwise. Thus, for example, reference to “a filament” can include two or more such filaments unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, “stratum corneum” refers to the outermost layer of the skin, consisting of from about 15 to about 20 layers of cells in various stages of drying out. The stratum corneum provides a barrier to the loss of water from inside the body to the external environment and from attack from the external environment to the interior of the body.

As used herein, “tissue” refers to an aggregate of cells of a particular kind, together with their intercellular substance, that forms a structural material. In the context of drug delivery to or through such tissue, at least one surface of the tissue is accessible to the transdermal delivery modality (e.g., poration device and/or patch). The tissue is the skin for various poration delivery modalities described herein. Other tissues suitable for use with this disclosure include mucosal tissue and soft organs.

As used herein, the term, “interstitial fluid” is the clear fluid that occupies the space between the cells in the body.

As used herein, the term “biological fluid” is defined as a fluid originating from a biological organism, including blood serum or whole blood as well as interstitial fluid.

As used herein, a “tissue membrane” can be any one or more epidermal layers of a subject. For example, in one aspect, the tissue membrane is a skin layer that includes the outermost layer of the skin, i.e., the stratum corneum. In an alternative aspect, a skin layer can include one or more backing layers of the epidermis, commonly identified as stratum granulosum, stratum malpighii, and stratum germinativum layers. It will be appreciated by one of ordinary skill in the art that there is essentially little or no resistance to transport or to absorption of a permeant through the backing layers of the epidermis. Therefore, in one aspect, an at least one formed pathway in a skin layer of a subject is a pathway in the stratum corneum layer of a subject. Further, as used herein, “stratum corneum” refers to the outermost layer of the skin, typically containing from about 15 to about 20 layers of cells in various stages of drying out. The stratum corneum provides a barrier to the loss of water from inside the body to the external environment and from attack from the external environment to the interior of the body. Still further, as used herein, “tissue membrane” can refer to an aggregate of cells of a particular kind, together with their intercellular substance, that forms a structural material. In various embodiments at least one surface of the tissue membrane is accessible to one or more of the poration devices and/or permeant compositions described herein. As noted above, the tissue membrane for various poration delivery modalities is the skin. Other tissues suitable for use with such devices and compositions include mucosal tissue and soft organs.

As used herein, the term, “subcutaneous fluid” can include, without limitation, moisture, plasma, blood, one or more proteins, interstitial fluid, and any combination thereof. In one aspect, a subcutaneous fluid according to this description is a moisture source comprising water.

As used herein, “poration,” “microporation,” or any such similar term means the formation of a small hole or crevice (subsequently also referred to as a “micropore”) in or through the tissue or biological membrane, such as skin or mucous membrane, or the outer layer of an organism to lessen the barrier properties of this biological membrane for the passage of at least one permeant from one side of the biological membrane to the other for select purposes. Preferably the hole or “micropore” so formed is approximately 1-1000 microns in diameter and extends into the biological membrane sufficiently to break the barrier properties of the stratum corneum without adversely affecting the underlying tissues. It is to be understood that the term “micropore” is used in the singular form for simplicity, but that the microporation devices described herein may form multiple artificial openings. Poration could reduce the barrier properties of a biological membrane into the body for selected purposes, or for certain medical or surgical procedures. For the purposes of this application, “poration” and “microporation” are used interchangeably and mean the same thing.

A “microporator” or “porator” is a component for a microporation device capable of microporation. Examples of a microporator or porator include, but are not limited to, a filament capable of conductively delivering thermal energy via direct contact to a biological membrane to cause the ablation of some portion of the membrane deep enough to form a micropore, an optically heated topical dye/absorber layer, an electromechanical actuator, a microlancet, an array of microneedles or lancets, a sonic energy ablator, a laser ablation system, a high-pressure fluid jet puncturer, and the like. As used herein, “microporator” and “porator” are used interchangeably.

As used herein “penetration” means the controlled removal of cells caused by the thermal and kinetic energy released when the pyrotechnic element explodes which causes cells of the biological membrane and possibly some adjacent cells to be “blown away” from the site. As used herein, “fusible” and “fuse” refer to an element that could remove itself from and electrical circuit when a sufficient amount of energy or heat has been applied to it. i.e., when a resistive, electrically activated poration element is designed to be a fusible element this means that upon activation, during or after the formation of the micropore in the biological membrane, the element breaks, stopping the current flow through it.

As used herein, “penetration enhancement” or “permeation enhancement” means an increase in the permeability of the biological membrane and/or tissue to a drug, bio-active composition, or other chemical molecule, compound, particle or substance (also called “permeant”), so as to increase the rate at which the drug, bio-active composition, or other chemical molecule, compound or particle permeates the biological membrane and/or tissue.

As used herein, “enhancer,” “chemical enhancer,” “penetration enhancer,” “permeation enhancer,” and the like includes all enhancers that increase the flux of a permeant, analyte, or other molecule across the biological membrane, and is limited only by functionality. In other words, all cell envelope disordering compounds and solvents and any other chemical enhancement agents are intended to be included. Additionally, all active force enhancer technologies such as the application of sonic energy, mechanical suction, pressure, or local deformation of the tissues, iontophoresis or electroporation are included. One or more enhancer technologies may be combined sequentially or simultaneously. For example, a chemical enhancer may first be applied to permeabilize the capillary wall and then an iontophoretic or sonic energy field may be applied to actively drive a permeant into those tissues surrounding and comprising the capillary bed.

As used herein, “transdermal” or “percutaneous” means passage of a permeant into and through the biological membrane to achieve effective therapeutic blood levels or local tissue levels of a permeant, or the passage of a molecule or fluid present in the body (“analyte”) out through the biological membrane so that the analyte molecule maybe collected on the outside of the body.

As used herein, the term “permeant,” “drug,” “permeant composition,” or “pharmacologically active agent” or any other similar term are used interchangeably to refer to any chemical or biological material or compound suitable for transdermal administration by the methods previously known in the art and/or by the methods taught in the present description, that induces a desired biological or pharmacological effect, which may include but is not limited to (1) having a prophylactic effect on the organism and preventing an undesired biological effect such as an infection, (2) alleviating a condition caused by a disease, for example, alleviating pain or inflammation, and/or (3) either alleviating, reducing, or completely eliminating the disease from the organism. The effect may be local, such as providing for a local anesthetic effect, or it may be systemic. Such substances include broad classes of compounds normally delivered into the body, including through body surfaces and membranes, including skin. In general, for example and not meant to be limiting, such substances can include any bioactive agents such as drug, chemical, or biological material that induces a desired biological or pharmacological effect. To this end, in one aspect, the permeant can be a small molecule agent. In another aspect, the permeant can be a macromolecular agent.

In various embodiments, systems, devices, and methods that may be used and/or adapted for use with the compositions and methods described herein are described in one or more of U.S. Pat. Nos. 6,022,316, 6,142,939, 6,173,202, 6,183,434, 6,508,785, 6,527,716, 6,692,456, 6,730,028, 7,141,034, 7,392,080, 7,758,561, 8,016,811, 8,116,860, and/or 9498609, all of which are hereby incorporated by reference in their entireties and particularly for the purpose of describing such systems and methods. In various embodiments, the systems and devices commercially available from PASSPORT® may be used or adapted for use in delivering the compositions described herein.

As used herein, an “effective” amount of a pharmacologically active agent means a sufficient amount of a compound to provide the desired local or systemic effect and performance at a reasonable benefit/risk ratio attending any medical treatment. An “effective” amount of a permeation or chemical enhancer as used herein means an amount selected so as to provide the desired increase in biological membrane permeability, the desired depth of penetration, rate of administration, and amount of drug delivered.

As used herein, “animal” or “organism” refers to humans and other living organisms including plants, to which the present disclosure may be applied.

As used herein, “analyte” means any chemical or biological material or compound suitable for passage through a biological membrane by the technology taught in this present disclosure, or by technology previously known in the art, of which an individual might want to know the concentration or activity inside the body. Glucose is a specific example of an analyte because it is a sugar suitable for passage through the skin, and individuals, for example those having diabetes, might want to know their blood glucose levels. Other examples of analytes include, but are not limited to, such compounds as sodium, potassium, bilirubin, urea, ammonia, calcium, lead, iron, lithium, salicylates, and the like.

As used herein, “transdermal flux rate” is the rate of passage of any analyte out through the skin of an individual, human or animal, or the rate of passage of any permeant, drug, pharmacologically active agent, dye, or pigment in and through the skin of an organism.

As used herein, “non-invasive” means not requiring the entry of a needle, catheter, or other invasive medical instrument into a part of the body.

As used herein. “minimally invasive” refers to the use of mechanical, hydraulic, or electrical means that invade the stratum corneum to create a small hole or micropore without causing substantial damage to the underlying tissues.

As used herein, “pharmaceutically acceptable carrier” refers to a carrier in which a substance such as a pharmaceutically acceptable drug could be provided for deliver. Pharmaceutically acceptable carriers are described in the art, for example, in “Remington: The Science and Practice of Pharmacy,” Mack Publishing Company, Pennsylvania, 1995, the disclosure of which is incorporated herein by reference. Carriers could include, for example, water and other aqueous solutions, saccharides, polysaccharides, buffers, excipients, and biodegradable polymers such as polyesters, polyanhydrides, polyamino acids, liposomes and mixtures thereof.

As used herein, “reservoir” refers to a designated area or chamber within a device which is designed to contain a permeant for delivery through an artificial opening in a biological membrane into an organism or may be designed to receive a biological fluid sample extracted from an organism through an artificial opening in a biological membrane. A reservoir may also contain excipient compounds which enhance the effect of a separately contained bioactive permeant. Additionally, a reservoir may contain or be treated with reactive enzymes or reagents designed to allow the measurement or detection of a selected analyte in an extracted biological fluid. A reservoir may be comprised of an open volume space, a gel, a flat planar space which has been coated or treated with a selected compound for subsequent release or reaction, or a matrix or permeable solid structure such as a pellet, tablet, powder, dried solid or porous polymer.

As used herein, “p3-Alcβ” is to be produced as metabolites of membrane protein Alcadein β (Alcβ). The Alep is metabolized by a pathway like that of Aβ to produce p3-Alcβ. Unlike Aβ that accumulates in the brain, p3-Alcβ decreases more significantly in Alzheimer's disease patients. The action of p3-Alcβ is carried out by its partial peptide, such as p3-Alcβ1-40, p3-Alcβ1-37, p3-Alcβ9-19, p3-Alcβ 1-19, p3-Alcβ11-19 or its derivatives.

### Patch

In aspects, the systems, devices and methods of the present disclosure can be used to transdermally deliver peptides across the skin. In some aspects, the patch may comprise a top layer including an adhesive, a middle layer including a matrix, and a bottom layer. In some embodiments, the bottom layer includes a release liner. In some embodiments, the middle layer further includes a PK modifier. In some embodiments, the middle layer further includes an enhancer. In some embodiments, the patch includes a tissue interface layer. In some embodiments, the patch further includes a backing. In some embodiments, the release liner is configured to be removed before application to the subject's skin. In some embodiments, the patch is configured as a film. In some embodiments, the patch is configured as a pellet.

Examples of suitable tissue interface layers are described in U.S. Pat. No. 7,392,080, which is hereby incorporated herein by reference in its entirety and particularly for the purpose of describing transdermal drug delivery patch systems.

In some embodiments, the top layer includes a backing. In some embodiments, the backing is a film, form, woven, or non-woven material. In some embodiments, the film includes a polyethylene (PE), polyethylene terephthalate (PET), polyurethane (PU), polyvinyl chloride (PVC), polychlorotrifluoroethylene (PCTFE), cyclic olefin copolymers (COC), or polymers (COP). In some embodiments, the backing includes adhesive. In some embodiments, the adhesive is an acrylic, a silicone or a synthetic rubber such as Polyisobutylene (PIB) and Styrene-Isoprene-Styrene block copolymer (SIS). In some embodiments, the color of backing is transparent, semi-transparent, tan, white, or beige. In some embodiments, the backing is formed by thermoforming to make a cavity. In some embodiments, the backing is covered by adhesive tape.

In some embodiments, the bottom layer includes a release liner. In some embodiments, the release liner is a film. In some embodiments, the film is polyethylene terephthalate, polyethylene, paper, or aluminum foil. In some embodiments, the film includes a silicone or fluorosilicone coated layer. In some embodiments, the release liner is heat-sealed to the backing film.

In some embodiments, the PK modifier is a drug delivery modifier. In some embodiments, the PK modifier is pH control agent. In some embodiments, the PK modifier is an organic acid, a salt form of the organic acid or a combination of thereof. In some embodiments, the organic acid is selected from ascorbic acid, citric acid, succinic acid, tartaric acid, maleic acid, lactic acid, benzoic acid, sorbic acid, amino acids, or a combination thereof. In some embodiments, the PK modifier is a non-organic acid. In some embodiments, the PK modifier is a salt form of the non-organic acid. In some embodiments, the non-organic acid is hydrochloric acid, phosphoric acid, boric acid, acetic acid or a combination thereof. In some embodiments, the non-organic acid is evaporated during the manufacturing process. In some embodiments, the organic base is selected from sodium citrate, Tris, mono-sodium phosphate, di-sodium phosphate, tri-sodium phosphate, mono-potassium phosphate, di-potassium phosphate, tri-potassium phosphate, basic amino acids, or a combination thereof.

In some aspects, the enhancer is a saccharide. In some embodiments, the saccharide comprises or is a sugar. In some embodiments, the enhancer is a non-reducing sugar. In some embodiments, the enhancer is a reducing sugar. In some embodiments, the saccharide is selected from mannitol, maltose, trehalose, xylitol, xylose, dextrose, lactose, sorbitol, sucrose, fructose, maltitol, erythritol, lactitol, isomalt, and cyclodextrin or a combination thereof. In some embodiments, the enhancer is sucrose. In some embodiments, the enhancer is lactose. In some embodiments, the enhancer is maltose. In some embodiments, the enhancer is a combination of sucrose and lactose.

In some embodiments, the weight ratio of enhancer to non-aggregating peptide is greater than 0.005, greater than 0.02, greater than 0.05, greater than 0.1, greater than 0.2, or ranges including and/or spanning the aforementioned values. In some embodiments, the weight ratio of a sugar to non-aggregating peptide is greater than 0.02. In some embodiments, the weight ratio of a sugar to non-aggregating peptide is from about 0.02 to about 10. In some embodiments, the weight ratio of a sugar to non-aggregating peptide is from about 0.02 to about 0.4.

In some aspects, the middle layer of the patch includes a reservoir. In some embodiments, the reservoir includes about 1.0% to about 99.5% by weight non-aggregating peptide. In some embodiments, the non-aggregating peptide includes approximately 50 weight % to approximately 98 weight % of the middle layer, including amounts such as 50 weight %, 55 weight %, 70 weight %, 80 weight %, 90 weight %, 95 weight %, 98 weight % of the middle layer, including any range of weight percentages derived from these values.

In some embodiments, the middle layer includes about 0.5% to about 99% by weight of an enhancer. In some embodiments, the enhancer includes approximately 2 weight % to approximately 50 weight % of the middle layer, including amounts such as 2 weight %, 5 weight %, 10 weight %, 15 weight %, 30 weight %, 40 weight %, 50 weight % of the middle layer, and including any range of weight percentages derived from these values.

In some embodiments, the middle layer includes about 5 to about 60% by weight of a PK modifier. In some embodiments, the PK modifier includes approximately 10 weight % to approximately 40 weight % of the middle layer, including additional amounts as 7.5 weight %, 15 weight %, 25 weight %, 40 weight % of the middle layer, and including any range of weight percentages derived from these values.

In some embodiments, the middle layer includes a PK modifier in an area amount of at least 0.5 mg/cm², at least 1 mg/cm², at least 2 mg/cm², at least 4 mg/cm², at least 8 mg/cm², or ranges including and/or spanning the aforementioned values, based on the surface area of the middle layer facing the bottom layer. In some embodiments, the middle layer includes at least 2 mg/cm² of disodium citrate. In some embodiments, the middle layer includes at least 4 mg/cm² of disodium citrate.

In some aspects, the middle layer includes a matrix support. In some embodiments, the matrix support includes at least one fiber. In some embodiments, the fiber is a nonwoven material. In some embodiments, the matrix support is a non-woven fabric. In some embodiments, the non-woven fabric is a polyethylene terephthalate. In some embodiments, the matrix support is a laminated material of film. In some embodiments, the film is a polyethylene terephthalate. In some embodiments, the matrix support is a laminated material of fiber. In some embodiments, the matrix is a laminated material of film and fiber.

In some embodiments, the thickness of the matrix support is less than 300 μm, less than 250 μm, less than 200 μm, less than 150 μm, less than 100 μm, less than 50 μm, or ranges including and/or spanning the aforementioned values.

In some embodiments, the areal weight of the fiber is less than 100 g/m², less than 90 g/m², less than 80 g/m², less than 70 g/m², less than 60 g/m², less than 50 g/m², less than 40 g/m², less than 30 g/m², less than 20 g/m², less than 10 g/m², or ranges including and/or spanning the aforementioned values.

In some embodiments, the matrix support has a water holding capacity (WHC) of from about 0.1 mg/cm² to about 10 mg/cm², based on the surface area of the matrix support facing the bottom layer. The water holding capacity of the matrix support means the amount of moisture the matrix support can hold per 1 cm² of the transdermal surface. Specifically, a 1 cm² matrix is prepared, and this is immersed in a solution (phosphate buffered saline containing 0.1% surfactant (Tween® 80)) for a sufficiently long amount of time. Following this, the matrix support is slowly pulled out of the solution for around five seconds, the weight of the sample before immersion measured in advance is subtracted from the weight of the sample holding the liquid, and then it is possible to determine the water holding capacity of the matrix per unit area (1 cm²) of the transdermal surface. In some embodiments, the matrix support water holding capacity is from about 10 mg/cm² or less. In some embodiments, the matrix support water holding capacity is from about 1 mg/cm² to about 8 mg/cm². In some embodiments, the matrix water holding capacity is from about 2 mg/cm² to about 5 mg/cm².

The water holding capacity of the matrix support may be controlled by adjusting the thickness and weight of the matrix support. It is preferable that the matrix support has a thickness of 100 μm or less. In some embodiments, the matrix support has a thickness in the range of about 10 μm to about 100 μm. In some embodiments, the matrix support thickness is about 20 μm to about 90 μm. In some embodiments, the matrix support thickness is about 30 μm to about 80 μm. In some embodiments, the matrix support thickness is about 40 μm to about 60 μm.

In some embodiments, the matrix support areal weight is about 10 g/m² to about 100 g/m². In some embodiments, the matrix support areal weight is about 15 g/m² to about 80 g/m². In some embodiments, the matrix support areal weight is about 20 g/m² to about 60 g/m². In some embodiments, the matrix support areal weight is about 25 g/m² to about 40 g/m².

In some embodiments, the matrix support areal weight is about 0.1 mg/cm² to about 30 mg/cm². In some embodiments, the matrix support areal weight is about 0.5 mg/cm² to about 30 mg/cm². In some embodiments, the matrix a support real weight is about 0.5 mg/cm² to about 20 mg/cm². In some embodiments, the matrix support areal weight is about 0.5 mg/cm² to about 10 mg/cm².

In some embodiments, the size of the matrix is about 0.125 cm² to about 4 cm². In some embodiments, the size of the matrix is about 0.25 cm² to about 3 cm². In some embodiments, the size of the matrix is about 0.5 cm² to about 2 cm². In some embodiments, the size of the matrix is about 0.5 cm². In some embodiments, the size of the matrix is about 1 cm². In some embodiments, the size of the matrix is about 2 cm².

In some embodiments, the total amount of non-aggregating peptide and enhancer per unit area of the matrix (surface area) is 0.01 mg/cm² to 200 mg/cm². In some embodiments, the total amount of non-aggregating peptide and enhancer per unit area of the matrix is 0.1 mg/cm² to 100 mg/cm². In some embodiments, the total amount of non-aggregating peptide and enhancer per unit area of the matrix is 5 mg/cm² to 75 mg/cm². In some embodiments, the total amount of non-aggregating peptide and enhancer per unit area of the matrix is 10 mg/cm² to 50 mg/cm².

In some embodiments, the pH of the matrix ingredients is from about 3 to about 9. In some embodiments, the pH of the matrix is from about 4 to about 8. In some embodiments, the pH of the matrix is about 4. In some embodiments, the pH of the matrix is about 5. In some embodiments, the pH of the matrix is about 6. In some embodiments, the pH of the matrix is about 7.

In some aspects, the matrix includes from about 0.01 mg/cm² to about 200 mg/cm² non-aggregating peptide, based on the surface area of the matrix facing the bottom layer. In some embodiments, the matrix includes from about 0.1 mg/cm² to about 100 mg/cm² non-aggregating peptide. In some embodiments, the matrix includes from about 1 mg/cm² to about 50 mg/cm² non-aggregating peptide. In some embodiments, the matrix includes from about 5 mg/cm² to about 30 mg/cm² non-aggregating peptide. In some embodiments, the matrix includes from about 5 mg/cm² to about 25 mg/cm² non-aggregating peptide. In some embodiments, the matrix includes about 0.5 mg/cm² non-aggregating peptide. In some embodiments, the matrix includes about 1 mg/cm² non-aggregating peptide. In some embodiments, the matrix includes about 2 mg/cm² non-aggregating peptide. In some embodiments, the matrix includes about 3 mg/cm² non-aggregating peptide. In some embodiments, the matrix includes about 5 mg/cm² non-aggregating peptide. In some embodiments, the matrix includes about 10 mg/cm² non-aggregating peptide. In some embodiments, the matrix includes about 20 mg/cm² non-aggregating peptide. In some embodiments, the matrix includes about 25 mg/cm² non-aggregating peptide. In some embodiments, the matrix includes about 50 mg/cm² non-aggregating peptide. In some embodiment, the non-aggregating peptide is p3-Alcβ. In some embodiments, p3-Alcβ is selected from at least one of p3-Alcβ1-40, p3-Alcβ1-37, p3-Alcβ9-19, p3-Alcβ1-19, p3-Alcβ11-19 or its derivatives. In some embodiments, the p3-Alcβ is p3-Alcβ9-19 or its derivatives.

In some aspects, the matrix includes from about 0.05 mg/cm² to about 100 mg/cm² enhancer, based on the area of the matrix facing the bottom layer. In some embodiments, the matrix includes from about 0.1 mg/cm² to about 50 mg/cm² enhancer. In some embodiments, the matrix includes from about 0.2 mg/cm² to about 5.0 mg/cm² enhancer. In some embodiments, the matrix includes from about 0.5 mg/cm² to about 10 mg/cm² enhancer. In some embodiments, the matrix includes from about 0.5 mg/cm² to about 4.0 mg/cm² enhancer. In some embodiments, the matrix includes from about 0.5 mg/cm² to about 2.0 mg/cm² enhancer. In some embodiments, the matrix includes about 0.5 mg/cm² enhancer. In some embodiments, the matrix includes about 1 mg/cm² enhancer. In some embodiments, the matrix includes about 2 mg/cm² enhancer. In some embodiments, the enhancer is at least selected from sucrose, lactose and maltose.

In some aspects, the patch further includes an anti-microbial agent. In some embodiments, the anti-microbial agent is selected from benzoic acid, methylparaben, propylparaben, benzalkonium chloride, chlorhexidine, cresol, salicylic acid, sorbic acid, sodium benzoate, benzetonium chloride and combinations thereof.

In some embodiments, the patch is configured as a dry patch formulation. In some embodiments, the dry patch includes a backing, a matrix including a non-aggregating peptide in a dry state, and a release liner. In some embodiments, the matrix further includes a PK modifier in a dry state. In some embodiments, the matrix further includes an enhancer in a dry state. In some embodiments, the matrix further includes an anti-microbial agent in a dry state. In some embodiments, the dry patch is a heat dried film manufactured by dispensing or casting process. In some embodiments, the dry patch is a tablet or pellet manufactured by compressed process. In some embodiments, the dry patch formulation includes p3-Alcβ9-19, sucrose, lactose, disodium citrate sesquihydrate, methylparaben, propylparaben, sodium benzoate, and benzalkonium chloride. In some embodiments, the dry state includes less than 10% of water in formulation. In some embodiments, the dry state includes less than 5% of water in formulation. In some embodiments, the dry state includes less than 2% of water in formulation. In some embodiments, the dry state includes less than 1% of water in formulation.

In some embodiments, the patch is configured as a lyophilized patch formulation. In some embodiments, the lyophilized patch includes a backing, a matrix including p3-Alcβ9-19 in a lyophilized state, and a release liner. In some embodiments, the lyophilized patch consists of a cavity with spacer. In some embodiments, the lyophilized patch consists of a blister packaging. In some embodiments, the matrix further includes a drug delivery modifier in a lyophilized state. In some embodiments, the matrix includes an enhancer in a lyophilized state. In some embodiments, the matrix further includes an anti-microbial agent in a lyophilized state. In some embodiments, the lyophilized patch is lyophilized by processes known to those skilled in the art. In some embodiments, the lyophilized patch is a tablet or pellet manufactured by compressed process. In some embodiments, the lyophilized patch formulation includes p3-Alcβ9-19, sucrose, lactose, disodium citrate sesquihydrate, methylparaben, propylparaben, sodium benzoate, and benzalkonium chloride. In some embodiments, the lyophilized patch includes less than 10% of water in formulation. In some embodiments, the lyophilized patch includes less than 5% of water in formulation. In some embodiments, the lyophilized patch includes less than 2% of water in formulation. In some embodiments, the lyophilized patch includes less than 1% of water in formulation. In some embodiments, the lyophilized patch includes less than 0.5% of water in formulation. In some embodiments, the lyophilized patch includes less than 0.1% of water in formulation.

In some embodiments, the patch is configured as a tablet patch. In some embodiments, the tablet patch is a thin solid tablet. Thin solid tablets can be in various wafer- or plate-like shapes such as elliptical, circular, triangular, rectangular, square, pentagonal, hexagonal, irregular, etc. In various embodiments thin solid tablets are substantially flat. In an embodiment, a substantially flat thin solid tablet is slightly curved or bowed to a degree that facilitates handling, e.g., as compared to a flat thin solid tablet that is more difficult to pick up from a flat surface. In various embodiments, a thin solid tablet as described herein has an area density of more than 30 mg/cm², more than 40 mg/cm², more than 50 mg/cm², more than 60 mg/cm², more than 70 mg/cm², more than 80 mg/cm², more than 90 mg/cm², or more than 100 mg/cm²; less than 400 mg/cm², less than 350 mg/cm², less than 300 mg/cm², less than 250 mg/cm², or less than 200 mg/cm²; or in any range having endpoints defined by any two of the aforementioned values. For example, in various embodiments, the thin solid tablet has an area density of more than 30 mg/cm² and less than 400 mg/cm²; more than 40 mg/cm² and less than 400 mg/cm²; or more than 30 mg/cm² and less than 400 mg/cm².

In various embodiments, a thin solid tablet as described herein has a thickness (depending on the area density and the area of a face) of about 0.01 mm or greater, about 0.02 mm or greater, about 0.03 mm or greater, about 0.04 mm or greater, about 0.05 mm or greater, about 0.05 mm or greater, about 0.1 mm or greater, about 0.2 mm or greater, about 0.5 mm or greater, or about 1 mm or greater; about 10 mm or less, about 5 mm or less; about 2 mm or less; or about 1 mm or less; or in any range having endpoints defined by any two of the aforementioned values. For example, in various embodiments, the thin solid tablet has a thickness in the range of about 0.01 mm to about 10 mm or in the range of about 0.1 mm to about 5 mm.

In various embodiments, the thin solid tablet has a face in a manner analogous to the front or back face of a coin. In various embodiments, a face of the thin solid tablet has an area of about 0.01 cm² or greater, about 0.05 cm² or greater, about 0.1 cm² or greater, about 0.25 cm² or greater, about 0.5 cm² or greater, about 0.75 cm² or greater, or about 1 cm² or greater; or about 50 cm² or less, about 25 cm² or less, about 15 cm² or less, about 10 cm² or less, about 5 cm² or less, or about 2 cm² or less, or in any range having endpoints defined by any two of the aforementioned values. For example, in various embodiments, a face of a thin solid tablet has an area in the range of about 0.01 cm² to about 25 cm², about 0.1 cm² to about 10 cm², or about 0.15 cm² to about 5 cm².

In some embodiments, the patch is configured as a reservoir patch. In some embodiments, the reservoir patch includes a backing, a spacer to make a cavity, a matrix including p3-Alcβ9-19 in a cavity, and a release liner. The matrix can be in various shapes such as elliptical, circular, triangular, rectangular, square, pentagonal, hexagonal, irregular, etc. In an embodiment, the depth of cavity is between about 0.5 mm and 10 mm. In some embodiments, the depth of cavity is between about 0.5 mm and 5 mm. In some embodiments, the depth of cavity is between 1 mm and 3 mm.

In some embodiments, the patch is configured as a lyophilized dry patch. In some embodiments, the lyophilized dry patch includes a backing, a middle layer comprising a matrix including lyophilized p3-Alcβ9-19, and a release liner. The matrix can be in various shapes such as elliptical, circular, triangular, rectangular, square, pentagonal, hexagonal, irregular, etc. In an embodiment, the matrix is between about 0.5 mm and 10 mm. In some embodiments, the matrix is between about 0.5 mm and 5 mm. In some embodiments, the matrix is between 1 mm and 3 mm.

In some embodiments, the patch is configured as a tablet patch. In some embodiments, the tablet patch includes a backing, a middle layer comprising a tablet including p3-Alcβ9-19, and a release liner. The matrix can be in various shapes such as elliptical, circular, triangular, rectangular, square, pentagonal, hexagonal, irregular, etc. In an embodiment, the matrix is between about 0.5 mm and 10 mm. In some embodiments, the tablet is between about 0.5 mm and 5 mm. In some embodiments, the tablet is between 1 mm and 3 mm.

In some embodiments, the patch is configured as a solid dispersed dry patch. In some embodiments, the solid dispersed dry patch includes a backing, a middle layer comprising a matrix including dry state p3-Alcβ9-19, and a release liner. The matrix can be in various shapes such as elliptical, circular, triangular, rectangular, square, pentagonal, hexagonal, irregular, etc. In an embodiment, the matrix is between about 0.5 mm and 10 mm. In some embodiments, the tablet is between about 0.5 mm and 5 mm. In some embodiments, the matrix is between 1 mm and 3 mm.

### Method/Uses

Aspects disclosed herein relate to administering to a subject in need an effective amount of a non-aggregating peptide as disclosed elsewhere herein. Some embodiments pertain to treating Alzheimer's disease through administration of a patch as disclosed herein. In some embodiments, the patch of the present disclosure may deliver a non-aggregating peptide into a subject transdermally through one or more micropores formed by a transdermal delivery system as described herein. In some embodiments, the method may include perforating the outermost stratum corneum and epidermis, and then applying a patch thereon, such that the non-aggregating peptides in the patch pass through the epidermis, diffuse into the capillary dermis, and enter systemic circulation. The non-aggregating peptides absorbed from skin may be avoided first-pass metabolism in liver and effectively delivered to the brain. Thus, embodiments of a patch of the present disclosure may provide drug administration means to replace injections, which can be suitably used for immediate release applications or sustained release of non-aggregating peptides, with a higher bioavailability compared to oral, nasal or passive transdermal administration.

In some embodiments, the patch of the present disclosure can also be used in a method for delivering a non-aggregating peptide through a target biological membrane, wherein the method includes a step for forming one or more micropores on a biological membrane, and a step for placing a patch so as to be in physical contact with the one or more micropores, such that at least a portion of the drug is soluble in biological moisture received from the target through the one or more micropores.

In some embodiments, a method of treating a subject includes: identifying a subject having a brain disease or condition; opening a plurality of micropores in the skin of the subject; and applying the patch to the subject's skin over the micropores for a period of time effective to result in transdermal delivery of the non-aggregating peptide. In some embodiments, the patch includes a top layer including an adhesive, a middle layer including a non-aggregating peptide, and a bottom layer. In some embodiments, the bottom layer includes a release liner. In some embodiments, the period of time is selected to deliver a therapeutically effective amount of the non-aggregating peptide through the plurality of micropores.

In some embodiments, the opening of the plurality of micropores in the skin of the subject includes applying a transdermal microporation apparatus to the subject's skin. In some embodiments, the transdermal microporation apparatus includes a conductive member including an array of conductive filaments. In some embodiments, the transdermal microporation apparatus includes a conductive member including an array of conductive filaments. In some embodiments, the transdermal microporation opens the micropores by thermal tissue ablation. In some embodiments, the transdermal microporation creates micropores through the stratum corneum to the epidermis.

The patch of the present disclosure may be applied to a subject with a disease or condition where an immediate effect or a sustained effect is expected. In some embodiments, the patch and transdermal delivery of the non-aggregating peptide may have a PK profile comparable to subcutaneous injection. In some embodiments, the patch and transdermal delivery of the non-aggregating peptide may have a PK profile superior to intravenous and subcutaneous injection. In some embodiments, the patch and transdermal delivery of the non-aggregating peptide may have a PK profile with a bioavailability greater than 100%, greater than 200%, greater than 300%, greater than 400%, greater than 500%, greater than 750%, greater than 1000%, greater than 1500%, greater than 2000%, greater than 2500%, greater than 3000%, greater than 3500%, greater than 4000%, greater than 4500%, greater than 5000% compared to intravenous (IV) administration, or ranges including and/or spanning the aforementioned values. In some embodiments, the patch and transdermal delivery of the non-aggregating peptide may have a PK profile with a bioavailability from about 350% to about 4800% compared to IV.

In some embodiments, the patch and transdermal delivery of the non-aggregating peptide may provide an enhanced delivery of the non-aggregating peptide through the skin of the subject. In some embodiments, the patch and transdermal delivery of the non-aggregating peptide may provide a longer lasting delivery of the non-aggregating peptide to the subject as compared to IV or subcutaneous administrations. In some embodiments, the patch and transdermal delivery of the non-aggregating peptide area under the curve (AUC) in plasma for the non-aggregating peptide is about 1.0, 2.0, 3.0, 4.0, 5.0, 6.0, 7.0, 8.0, 9.0, 10.0 times higher than intravenous injections, or ranges including and/or spanning the aforementioned values. In some embodiments, the patch and transdermal delivery of the non-aggregating peptide area under the curve (AUC) in plasma for the non-aggregating peptide is 5.4 times higher than intravenous injections, the patch and transdermal delivery of the non-aggregating peptide area under the curve (AUC) in plasma for the non-aggregating peptide is 8.9 times higher than intravenous injections.

In some embodiments, the patch and transdermal delivery of an effective amount of the non-aggregating peptide into the blood of the subject is achieved at least 0.5 hour after administration, about 1 hour after administration, about 2 hours after administration, about 3 hours after administration, about 4 hours after administration, about 5 hours after administration, about 6 hours after administration, about 7 hours after administration, about 8 hours after administration, or ranges including and/or spanning the aforementioned values.

In some embodiments, the patch and transdermal delivery of an effective amount of the non-aggregating peptide into the blood of the subject was maintained more than 3 hours after administration, more than 4 hours after administration, more than 5 hours after administration, more than 6 hours after administration, more than 7 hours after administration, more than 8 hours after administration, more than 9 hours administration, more than 10 hours after administration, or ranges including and/or spanning the aforementioned values.

In some embodiments, the patch and transdermal delivery of the non-aggregating peptide into the cerebrospinal fluid achieves a Tmax about 1 hour after administration, about 2 hours after administration, about 3 hours after administration, about 4 hours after administration, about 5 hours after administration, about 5 hours after administration, or ranges including and/or spanning the aforementioned values.

In some embodiments, the transfer of the non-aggregating peptide from the blood to the cerebrospinal fluid (amount of non-aggregating peptide in the cerebrospinal fluid divided by the amount in the blood plasma, expressed as a percentage) is about 1.0%, about 1.5%, about 2.0%, about 3.0%, about 3.5%, about 4.0%, about 4.5%, about 5.0%, about 5.5%, about 6.0%, or ranges including and/or spanning the aforementioned values. In some embodiments, the transfer of the non-aggregating peptide from the blood to the cerebrospinal fluid is about 3.5%.

In some embodiments, a method is provided for intracerebral mitochondrial activation. In some embodiments, the method includes opening at least one micropathway in the subject's skin and applying a patch as disclosed herein to the subject's skin. In some embodiments, the BCPP-EF accumulation activation of mitochondria is achieved 0.5 hours after administration, 1.0 hours after administration, 1.5 hours after administration, 2.0 hours after administration, 2.5 hours after administration, 3.0 hours after administration, 3.5 hours after administration. 4.5 hours after administration. 5.0 hours after administration, or ranges including and/or spanning the aforementioned values. In some embodiments, the intracerebral mitochondrial activation is maintained more than 3 hours after administration, more than 4 hours after administration, more than 5 hours after administration, more than 6 hours after administration, more than 7 hours after administration, more than 8 hours after administration, more than 9 hours administration, more than 10 hours after administration, or ranges including and/or spanning the aforementioned values.

In some embodiments, a subject receives sufficient non-aggregating peptide from multiple dosages before high levels of non-aggregating peptides are achieved. One can readily and immediately envision a regimen wherein a subject is administered a first patch, and the subject receives one or more subsequent patches. Such a regimen may continue such that the subject receives a third patch after the subject receives the second patch. In some embodiments, a subject may receive: 1, 2, 3, 4, 5, 6, 7, 8, 9, 10 or more patches during treatment. One or more additional patches as described herein may be administered before the first patch dose, or before one or more subsequent patch dosages. In some embodiments, the subject receives doses over the time course of the remainder of his or her lifetime and/or over a period of years (e.g., greater than or equal to 1 year, 5 years, 10 years, 15 years, 20 years, 30 years, or ranges including and/or spanning the aforementioned values).

In some instances, a period of time passes between administering one or more patches to a subject. In some embodiments, the time period between one or more patches administered is equal to or at least about: twice daily, 1 day, 1 week, 2 weeks, 3 weeks, 1 month, 2 months, 3 months, 4 months, 5 months, 6 months, 7 months, 8 months, 9 months, 10 months, 11 months, 1 year, or ranges including and/or spanning the aforementioned values. In some embodiments, one or more additional therapeutic agents are administered to the subject during the period between the subject's administrations of the composition.

A subject in need of receiving a patch as disclosed herein to improve the subject's health need not always be identified prior to receiving a first treatment with the patch described herein. For example, a subject may be predetermined to develop Alzheimer's disease in the future, prior to showing any present signs or symptoms of Alzheimer's disease (cognitively normal at preclinical stage) or mild cognitive concerns of Alzheimer's disease (mild MCI stage). Alternatively, the subject may receive treatment prophylactically if he or she is at risk or not at risk of Alzheimer's disease (e.g., once a patient reaches an age of equal to or greater than 50, 60, 70, etc.). Accordingly, in some embodiments, the patch is administered to the subject after the subject receives an early stage diagnosis. In some embodiments, not every subject is a candidate for such administration and identification of treatment subjects may be desirable. It is understood that patient selection depends upon a number of factors within the skill of the ordinarily skilled physician. Thus, some embodiments disclosed herein further comprise identifying a subject as one that will benefit from administering an effective amount of at least one non-aggregating peptide or composition including the same to increase longevity, increase survival time, increase life span, or improve upon immunization. Subjects may be identified on the basis of physiological factors specific to the subject according to the subject's age, present medical condition, present medical treatment, prescribed medical treatment, or in some embodiments, the subject being diagnosed with Alzheimer's disease. In some embodiments, treatment of Alzheimer's disease includes preventing, reducing, and/or slowing the accumulation of beta-amyloids, amyloid plaques, and/or tangles in tau proteins. Beta-amyloid is a leftover fragment of a larger protein. When these fragments cluster together, they appear to have a toxic effect on neurons and to disrupt cell-to-cell communication. These clusters form larger deposits called amyloid plaques, which also include other cellular debris. Tau proteins play a part in a neuron's internal support and transport system to carry nutrients and other essential materials. In Alzheimer's disease, tau proteins change shape and organize themselves into structures called neurofibrillary tangles. The tangles disrupt the transport system and are toxic to cells.

In some aspects, a method of treating, preventing, or ameliorating a brain disease by administering a patch as described herein. In some embodiments, the brain disease is dementia. In some embodiments, the dementia is caused by Alzheimer's and Parkinson's Diseases. Dementia is a broad category of brain disease that cause a long-term and often gradual decrease in the ability to think and remember that is severe enough to affect daily functioning. Symptoms associated with dementia include emotional problems, difficulties with language, and a decrease in motivation. In some embodiments, the brain disease is neurodegenerative diseases such as Amyotropic lateral aclerosis (ALS), Huntington's disease, Ataxia, Spinal muscular atrophy, Lewy body disease. In some embodiments, the method includes testing a subject for a dementia risk factor and administering a composition as described herein. In some embodiments, testing a subject for a dementia risk factor include performing a brain scan, and amyloid PET, performing a brain biopsy, cognitive testing, and testing a subject's blood. Subjects may be identified on the basis of physiological factors specific to the subject according to the subject's age, present medical condition, present medical treatment, prescribed medical treatment, or in some embodiments, the subject being diagnosed with dementia.

Some aspects provided herein provide for a method of producing supraphysiological high levels of a non-aggregating peptide in a subject. In some embodiments, the method includes providing a non-aggregating peptide, composition, or patch as disclosed elsewhere herein to a subject. In some embodiments, the method includes administering an effective amount of a non-aggregating peptide as disclosed herein. In some embodiments, as disclosed herein, the composition includes a non-aggregating peptide, an enhancer, a PK modifier, and an anti-microbial agent. In some embodiments, the enhancer is a reducing sugar. In some embodiments, the PK modifier is an organic acid. In some embodiments, the anti-microbial agent is parabens. In some embodiments, the anti-microbial agent is sodium benzoate. In some embodiments, the patch provides an increased bioavailability of the non-aggregating peptide by at least 200%. In some embodiments, the method provides a transfer rate of the non-aggregating peptide from the blood to the CSF by about 3.5%.

Some aspects provide a method of treating or ameliorating a disease, disorder, or condition associated with Alzheimer's Disease in a subject; the method including administering a therapeutically effective amount of a non-aggregating peptide, composition, or patch as disclosed elsewhere herein to a subject. In some embodiments, the patch includes a non-aggregating peptide, an enhancer, a PK modifier, and an anti-microbial agent. In some embodiments, the non-aggregating peptide or composition improves the subject's cerebral blood flow. In some embodiments, the aggregating peptide or composition prevents or inhibits the hyperphosphorylation of the tau protein. In some embodiments, the hyperphosphorylation of the tau protein is inhibited by equal to or at least about: 50%, 70%, 80%, 90%, 99%, or ranges including and/or spanning the aforementioned values. In some embodiments, the composition reduces 3-amyloids in the subject's brain. In some embodiments, the Q-amyloids in the subject's brain are reduced by equal to or at least about: 50%, 70%, 80%, 90%, 99%, or ranges including and/or spanning the aforementioned values. In some embodiments, the aggregating peptide or composition has neuroprotection and nerve activation.

In some embodiments, a treatment method as described herein downregulates β-secretase activity in a subject. In some embodiments, the β-secretase activity is reduced by equal to or at least about: 50%, 70%, 80%, 90%, 99%, or ranges including and/or spanning the aforementioned values. In some embodiments, a treatment method as described herein upregulates α-secretase activity in a subject. In some embodiments, the α-secretase activity is increased by equal to or at least about: 150%, 170%, 180%, 190%, 199%, or ranges including and/or spanning the aforementioned values. In some embodiments, a treatment method as described herein upregulates neprilysin activity in a subject. In some embodiments, the neprilysin activity is increased by equal to or at least about: 150%, 170%, 180%, 190%, 199%, or ranges including and/or spanning the aforementioned values.

In aspects, administering a composition described herein may increase longevity, survival time, life span, or health span of the subject. In some embodiments, the expected longevity, survival time, life span, or health span of the subject is the median expectation for similarly situated subjects. In other embodiments, the expected longevity, survival time, life span, or health span of the subject is the mean expectation for similarly situated subjects. Subjects of similar situation may be determined based upon any one or more factors, including but not limited to, age, health, family history, or activity levels. In some embodiments, the expected increase as measured from the time treatment is started may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 1000%, about any of the aforementioned percentages, or a range bounded by any of the aforementioned percentages (e.g., about 1%-30%, about 5%-25%, about 5%-20%, about 5%-15% or 1%-30%, 5%-25%, 5%-20%, 5%-15%), 1%-100%, 1%-90%, 1%-80%, 1%-70%, 1%-60%, 1%-50%, 1%-40%, 1%-30%, 1%-20%, 1%-10%, 10%-100%, 10%-90%, 10%-80%, 10%-70%, 10%-70%, 10%-60%, 10%-50%, 10%-40%, 10%-30%, 10%-20%, 20%-100%, 20%-90%, 20%-80%, 20%-70%, 20%-60%, 20%-50%, 20%-40%, 20%-30%, 30%-100%, 30%-90%, 30%-80%, 30%-70%, 30%-60%, 30%-50%, 30%-40%, 40%-100%, 40%-90%, 40%-80%, 40%-70%, 40%-60%, 40%-50%, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-1000%, about any of the aforementioned range of percentages (e.g., about 10%-70%, about 30%-60%, or about 50%-70%), relative to the expected longevity, survival time, life span, or health span of the subject. In some embodiments, the expected increase is in years and is 1-40 years, 1-19 years, 1-18 years, 1-17 years, 1-16 years, 1-15 years, 1-14 years, 1-13 years, 1-12 years, 1-11 years, 1-10 years, 1-9 years, 1-8 years, 1-7 years, 1-6 years, 1-5 years, 1-4 years, 1-3 years, 1-2 years, 1 year, at least the aforementioned years (e.g., at least 1-10 years), or about the aforementioned years (e.g., about 1-2 years or at least about 1-2 years), relative to the expected longevity, survival time, life span, or health span of the subject. In some embodiments, the expected increase is in days to months, and is one day to one year, one day to 11 months, one day to 10 months, one day to 9 months, one day to 8 months, one day to 7 months, one day to 6 months, one day to 5 months, one day to 4 months, one day to 3 months, one day to 2 months, one day to one month, at least the aforementioned range of days to months (e.g., at least one day to 11 months), or about the aforementioned range of days to months (e.g., about one day to 6 months or at least about one day to 6 months), relative to the expected longevity, survival time, life span, or health span of the subject.

Some embodiments pertain to treating a disease or condition associated with the brain in a subject. In some embodiments, the subject's β-amyloid 42/β-amyloid 40 ratio is lowered. In some embodiments, the composition improves the subject's brain glucose metabolism. In some embodiments, the composition improves the subject's cerebral blood flow. In some embodiments, the composition prevents or substantially prevents the hyperphosphorylation of the tau protein. In some embodiments, the composition reduces β-amyloids the subject's brain. In some embodiments, the composition reduces cardiac arrest and stroke risk in a subject.

Some embodiments pertain to increasing mitochondria activity of a subject's brain. In some embodiments, the mitochondria activity of the subject's brain may be increased by at least about 70%, by at least 75%, by at least 80%, by at least 85%, by at least 90%, by at least 95%, or ranges including and/or spanning the aforementioned values. In some embodiments, the mitochondria activity of the subject's brain may be restored. In some embodiments, the mitochondria activity of the subject's brain is restored to at least 70% of normal activity, to at least 75% of normal activity, to at least 80% of normal activity, to at least 85% of normal activity, to at least 90% of normal activity, to at least 95% of normal activity, or ranges including and/or spanning the aforementioned values.

In some embodiments, the method for treatment of cognitive impairment induced by amyloid β protein according to the present discloser may include a step of administration of a patch to a mammal (human, or a mammal other than human) which developed cognitive impairment induced by amyloid β protein or to a model animal (a mammal other than human) thereof. In some embodiments, the method for treatment of Alzheimer's disease according to the present disclosure, may include a step of administration a patch as described herein to a mammal (excluding human) which developed Alzheimer's disease or to a model animal thereof.

The cognitive impairment induced by amyloid β protein to be treated with the therapeutic agent according to the present disclosure typically means symptoms accompanying Alzheimer's disease, but may be symptoms accompanying a disease which has not been definitively diagnosed with Alzheimer's disease, or symptoms in a subject in the preclinical stage including mild cognitive impairment (MCI) or in a model animal. The model animal of cognitive impairment induced by amyloid β protein can be prepared using a known method. For example, a transgenic mouse which exhibits excessive expression of amyloid β protein or a mouse to which a solution of amyloid β protein in an artificial cerebrospinal fluid is administered can be used as such a model animal.

In some embodiments, as discussed elsewhere herein, the subject is a human. However, the methods are not limited to the treatment of humans and are equally applicable to the treatment of mammals. In such instances of treating non-human mammals, patient selection depends upon a number of factors within the skill of the ordinarily skilled veterinarian or research scientist.

### System/Apparatus

The basic configurations of microporation drug delivery systems are known to those skilled in the art and thus do not require further elaboration herein. For example, transdermal permeant delivery systems are described in U.S. Pat. No. 8,116,860, which is hereby incorporated herein by reference and particularly for the purpose of describing various features of such microporation drug delivery systems. As described therein, the microporation drug delivery system of U.S. Pat. No. 8,116,860 (referred to therein using the reference number “10”) includes basic features that include a filament array (referred to therein using the reference number “70”) configured to create the one or more pathways or micropores in a patient's skin and one or more transdermal patches (referred to therein using the reference number “100”) containing at least one drug formulation. Other similar microporation drug delivery systems including such basic features are known to those skilled in the art. Various microporation drug delivery systems having such basic features are known to those skilled in the art and may be used or adapted for use by those skilled in the art guided by the teachings provided herein.

In some embodiments, the microporation device may be defined by a total area of pathways (for example, micropores) created in the skin by the one or more filaments and a total energy delivered to the one or more filaments to create the pathways. In some embodiments, the microporation device creates pathways such that the total area of pathways in the skin is between approximately 0.25 and 4.0 of a square centimeter (cm) of the skin. In some embodiments, the pathways in the skin may preferably comprise approximately 0.5 to 12.5% of the skin area for each square centimeter of the skin exposed to the microporation device. In some embodiments, the pathways in the skin may preferably comprise approximately 1.25 to 10% of the skin area for each square centimeter of the skin exposed to the microporation device.

In some embodiments, the energy delivered to the one or more filaments to create the pathways may be in the range of 0.0067 μJ/μm³-0.0400 μJ/μm³. The energy delivered to the one or more filaments may be delivered in pulses of between 2 and 12 milliseconds (ms) for sufficient energy to create consistent pathways to be delivered. Characteristics of pathways that efficiently and safely deliver a drug through the skin, for example from a patch as described herein, may vary between in vitro and in vivo embodiments. For example, in some embodiments, the one or more filaments creates the pathway when energy of between 2 mJ/filament and 12 mJ/filament is applied to the one or more filaments for a pulse of between 2 and 16 ms. In some embodiments, the energy applied to the one or more filaments to create the pathway is between 2 mJ/filament and 8 mJ/filament, 2 mJ/filament and 6 mJ/filament or between 2 mJ/filament and 4 mJ/filament. In some embodiments, the duration of the pulse is between 2 and 12 ms. In such embodiments, the one or more filaments may comprise or substantially be formed from stainless steel and having a volume (V) of 300,000 μm³ (0.0067 μJ/μm³-0.0400 mJ/μm³) for 2 mJ/filament-12 mJ/filament.

In some embodiments, the one or more filaments, arranged in a filament array, can create between 25 and 500 pathways/cm² of the biological membrane (e.g., skin) to which the one or more filaments are exposed. In some embodiments, the one or more filaments arranged in a filament array, can create between 50 and 400 pathways/cm² of the skin to which the one or more filaments are exposed.

In some embodiments, an accumulated (or summed) depth of all the pathways formed by the one or more filaments is between approximately 2500 and 30000 μm per square centimeter of skin exposed to the one or more filaments. In some embodiments, an accumulated or summed volume of all the pathways formed by the one or more filaments is between approximately 0.05 and 0.35 mm³ per square centimeter of skin.

In some embodiments, the patch described herein may have one or more characteristics that enhance the optimal drug release and diffusion of the non-aggregating peptide into the body in conjunction with the pathways created by the microporation device.

In some embodiments, the improvements to the microporation device and the patch as described herein enable the microporation drug delivery system to effectively and safely provide delivery of the non-aggregating peptide in a manner that provides improved bioavailability and/or transferability of the non-aggregating peptide from the blood to the cerebrospinal fluid of the subject. Embodiments of the microporation drug delivery system described herein may provide for improved patient compliance and enhanced drug delivery capabilities. Embodiments of the microporation drug delivery system may also provide for reduced risks of adverse effects caused by uncontrolled delivery and reduce development terms and costs for drugs for patients. Embodiments of the microporation drug delivery system also enables painless and needle-free self-administration of corresponding drugs by the patient a location of patient's choice, which leads to improved compliance and reduced costs (less visits to health care professionals). Embodiments of the microporation drug delivery system as described herein may be used for patients with a wide range of skin types, conditions, and so forth with a lower variation on individual drug delivery results.

### EXAMPLES

Various embodiments and alternatives are disclosed in further detail in the following examples, which are not in any way intended to limit the scope of the claims.

Alzheimer's Disease. Alzheimer's Disease is a chronic disease that progresses with aging. Thus, due to the nature of Alzheimer's Disease, medication may require daily administration(s) for the long term. As such, IV administration is not desired because it would require hospitalization or daily visits. Subcutaneous administration may be developed as a self-administration device, such as a pen injector. However, it is believed that due to the patient's pain and problems with handling, it would not be efficient over the long term for most patients. In addition, the patient sometimes has a needle phobia. Thus, it is believed that transdermal microporation would have good compliance and would work as a long-term at-home therapy. In this study, transdermal microporation devices were investigated, compared to other modes of administration, for the non-aggregating peptide, p3-Alcβ, to enhance the delivery of p3-Alcβ to the brain and determine the effectiveness of the various modes of administration.

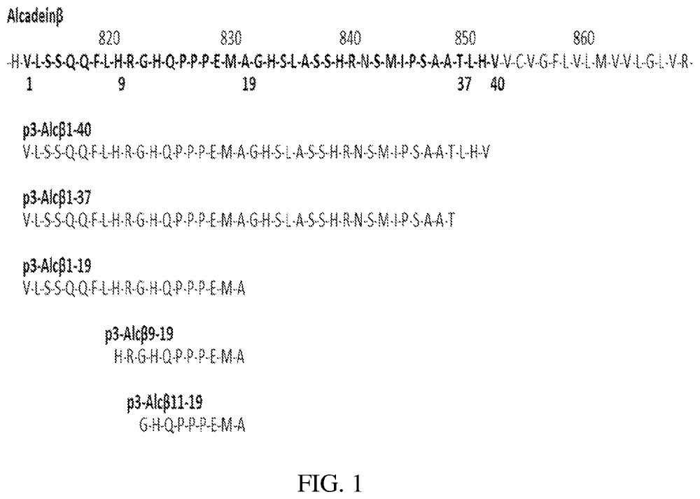

p3-Alep. FIG. 1 describes amino acid sequences of p3-Alcβ1-40, p3-Alcβ1-37, p3-Alcβ9-19, p3-Alcβ 1-19, p3-Alcβ1-19 which are the partial peptides of Alcadein β. Human p3-Alcβ37 peptide, which includes the sequence from Val813 to Thr849 of Alcβ described in Hata et al “Alcadein cleavages by amyloid β-precursor protein (APP) α- and γ-secretase generates small peptides, p3-Ales, indicating Alzheimer's disease-related γ-secretase dysfunction” J. Biol. Chem. [2009]284, 36024-36033, and its partial peptides, p3-Alcβ9-19, were synthesized and purified to greater than 95% purity; their expected molecular weights were confirmed by mass spectroscopy, performed at Peptide Institute (Osaka, Japan).

Animal Studies in Rats. Oral, nasal, injections, and transdermal microporation containing p3-Alcβ9-19 were tested to in vivo determine pharmacokinetics such as PK profile, bioavailability, and drug recovery. In vivo PK studies were performed in CD rats (Charles River Laboratories) according to IACUC-approved Animal Protocols. Each dosing formulation was prepared prior to the experiments. The patch was manufactured according to dry patch manufacturing procedure. The animals for transdermal applications were shaved 1 day before the dosing date. The transdermal microporation was applied using the Applicator and Porator. Then, the patch was placed to cover the porated area for 1 day or a designated duration. After the last blood sampling, the patch was collected, and the application site was wiped by paper or cotton to recover the remaining drug on the skin. The blood samples were collected from the tail vein and transferred into K2-EDTA tube at the designated sampling time. The samples were centrifuged at 4° C., and then plasma samples were obtained. All samples were stored at −80° C. freezer until bioassay.

Sample Preparations for LCMSMS analysis of p3-Alcβ9-19. The plasma samples were stayed at room temperature for 30 minutes. The plasma (50 μL) was transferred into a 2 mL Eppendorf tube (Protein LoBind), then add 50 μL of Acetonitrile/DMSO (4:1, v/v), and vortex the tube for a couple of seconds. The samples were centrifuged for 15 minutes at 3700 rpm, 4° C. The 150 μL of mobile phase A (0.1% formic acid in water) were put into a new tube, then 50 μL of the supernatant were transferred into the tube and the samples were vortexed for 5 minutes. The supernatant was pulled out and the solution was filtered through a 0.20 μm PTFE membrane into an autosampler vial (LCMSMS).

LCMSMS method of p3-Alcβ9-19. The LCMSMS conditions shows in Tables 1 and 2.

TABLE 1

| Parameter | | Condition | | |
|---|---|---|---|---|
| PDA Detector | | Open | | |
| Wavelength | | 200 nm | | |
| Column | | Agilent, ZORBAX Eclipse Plus C18 | | |
| | | 1.8 μm, 2.1 × 50 mm | | |
| Column Temperature | | 40° C. | | |
| Injection Volume | | 2 μL | | |
| Run Time | | 5.00 Minutes | | |
| Mobile Phase | Mobile | Water/formic acid (100:0.1, v/v) | | |
| | Phase A | | | |
| | Mobile | Acetonitrile/formic acid (100:0.1, v/v) | | |
| | Phase B | | | |
| Pump Mode | | Gradient | | |
| | Time | Flow | % MPA | % MPB |
| Gradient Pump | 0.00-2.50 | 0.35 mL/min | 97 → 80 | 3 → 20 |
| Mode Set-Up | 2.50-2.70 | 0.35 mL/min | 80 → 20 | 20 → 80 |
| | 2.70-3.50 | 0.35 mL/min | 20 | 80 |
| | 3.50-3.70 | 0.35 mL/min | 20 → 97 | 80 → 3 |
| | 3.70-5.00 | 0.35 mL/min | 97 | 3 |

TABLE 2

| Parameter | Condition | |
|---|---|---|
| Ion Spray Voltage | 5500 | V |
| Curtain Gas | 35 | psi |
| Ion Source Gas GS1/GS2 | 50/50 | psi |
| Collision Gas | High | |
| Temperature | 650° | C. |
| Analyte/Monitor Ion | 1028.390 > 383.000/514.290 > 318.300 | |
| Declustering Potential | 40 | V |
| Entrance Potential | 10 | V |
| Collision Energy | 20 | V |
| Collision Cell Exit Potential | 10 | V |
| Divert Valve | 1.0 to 2.7 min (introduced to MS/MS) | |
| Dwell Time | 150 | msec |

Animal Studies in Mice. The transport of p3-Alcβ9-19 into the blood and cerebrospinal fluid (CSF) were tested in wild-type mice (4 months). The hair was removed prior to the experiments. The transdermal microporation was applied, and then the patch containing p3-Alcβ9-19 was administered on the porated area. The mice were anesthetized by using 1% isoflurane and CSF was collected from the cisterna magna and blood was collected from the inferior vena cava in mice into the tube containing EDTA and heparin as described in Liu et al, “A technique for serial collection of cerebrospinal fluid from the cisterna magna in mouse.” J. Vis. Exp. 21 (2008).

sELISA method development for p3-Alcβ9-19 Analysis. sELISA method was developed to determine p3-Alcβ9-19 in cerebrospinal fluid (CSF). Polyclonal rabbit antibody to p3-Alcβ9-19 was raised against p3-Alcβ9-19 containing an amino-terminal Cys residue (C+HRGHQPPPEMA) and conjugated to bovine thyroglobulin. IgG was purified with antigen-coupled resin and conjugated to biotin. Horseradish peroxidase-conjugated streptavidine was from Amersham/GE Healthcare (Cat #RPN1051, Little Chalfont, UK), and the tetramethylbenzidine (TMB) microwell peroxidase substrate system was from SeraCare Life Sciences Inc. (Cat #5120-0075, Milford, MA, USA). The mice were anesthetized by using 1% isoflurane, and CSF was collected from the cisterna magna in mice as described above, and then mice were sacrificed.

To quantify p3-Alcβ9-19 in mouse CSF and plasma, the samples were diluted with buffer A (PBS containing 1% bovine serum albumin and 0.05% Tween-20). Using the polyclonal antibody, the developed sELISA system was quantified p3-Alcβ9-19 at a range of 25-200 μg/ml, a sensitivity equivalent to p3-Alcα35-specific, described in Omori et al., “Increased levels of plasma p3-Alcα35, a major fragment of Alcadeivα by γ-secretase cleavage, in Alzheimer's disease” J. Alzheimers. Dis. (2014) 39, 861-870, p3-Alcβ37- and p3-Alcβ40-specific sELISA systems, described in Hata et al “Decrease in p3-Alcβ37 and p3-Alcβ40, products of Alcadeinβ generated by γ-secretase cleavages, in aged monkeys and patients with Alzheimer's disease” Alzheimers Dement TRCI (2019) 5, 740-750. In this p3-Alcβ9-19 sELISA system, antiserum diluted 1:10,000 times was used as a capture antibody, and affinity-purified antiserum with antigen-coupled resin and biotin-labeled IgG was used for detection antibody. This sELISA system did not react with p3-Alcβ37. The addition of 1,000 pg/ml p3-Alcβ37 did not compete with antibody binding to 0-200 pg/ml p3-Alcβ9-19. The sELISA method was used for measuring p3-Alcβ9-19 in body fluids, even in the presence of endogenous p3-Alcβ 3 in mice.

sELISA method for p3-Alcβ9-19 Analysis. Rabbit antiserum immunized with p3-Alcβ9-19 as an antigen was coated on a 96-well plate as a capture antibody and overnight at 4° C. The next day, the non-binding antibody was removed with wash buffer (0.05% Tween-20 in PBS). The plasma and CSF were diluted 200-fold and 20-fold with EIA buffer (1% BSA, 0.05% Tween-20), respectively. The sample solution was added to 96 well plates and stayed overnight at 4° C. The next day, after washing with wash buffer, purified biotinylated IgG dissolved in PBS was added to 1.0 g/mL in each well, and the mixture was kept overnight at 4° C. The next day, the unreacted antibody was washed with wash buffer, streptavidin-HRP (1/5000 in PBS) was added, and the reaction was carried out at 4° C. for 6 to 8 hours. After washing with Wash buffer, TMB color-developing substrate was added, and the mixture was reacted at room temperature for 30 minutes under light-protection. The color development was stopped by adding 1 N H₂SO₄, and the samples were measured by a plate reader at 450 nm.

Intracerebral mitochondrial activation. The increase of neuronal viability by p3-Ale was corroborated in the in vivo setting by monitoring brain mitochondria function using PET imaging with [18F]BCPP-EF probe, which can detect mitochondrial complex I activity reflecting neuronal viability in the living brain. Mitochondrial dysfunction generally exists in the brain of AD patients, and the lowered viability of vulnerable brain regions is detectable by PET imaging with [18F]BCPP-EF.

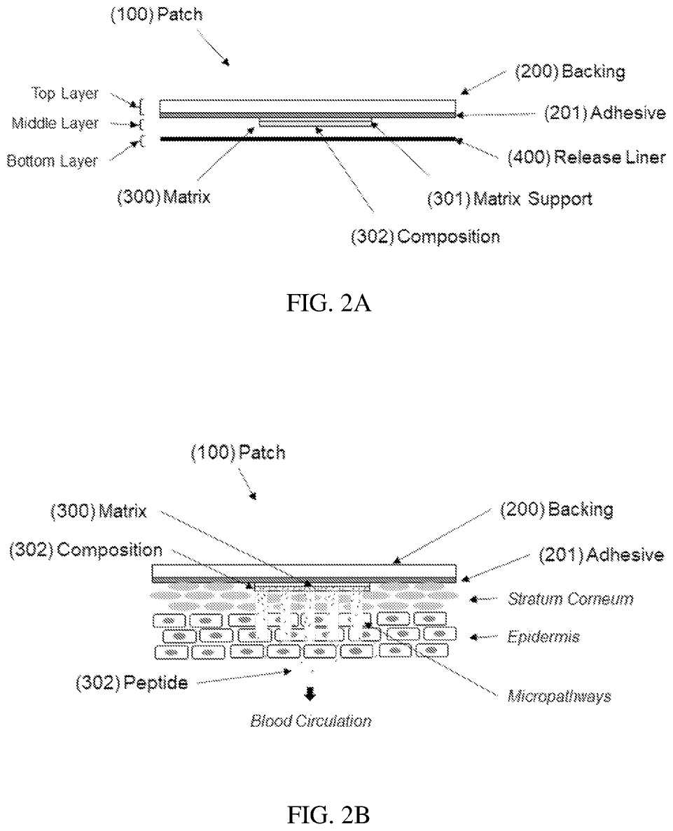

Transdermal Microporation Device. FIG. 2A describes an example of patch; FIG. 2B illustrates a patch application after transdermal microporation in skin. The microporation device consists of (a) an applicator electrically connected to the filament, (b) a porator having an upper substrate surface and defining a poration area, and (c) a patch. The filament array having a plurality of filaments are disposed in the poration area, and each filament is capable of conductively delivering thermal energy via direct contact to the tissue membrane to form at least one micropore in the tissue membrane. The applicator supplies a predetermined electrical energy to the filaments in order to create the micropore, and a patch is applied on the at least part of the micropore. The patch (100) consists of a backing (200), a matrix (300) containing a composition (302) such as p3-Alcβ and ingredients as a dry state, and a release liner (400) that is removed before application to porated skin. The backing (200) may have an adhesive (201). A matrix (300) may consist of matrix support (301) and composition (302) disposed to matrix support (301). After transdermal microporation, a patch (100) is applied on the porated skin after removing a release liner (400). The active ingredient in a composition (302) is dissolved by interstitial fluid from micropathways, then active ingredients migrate to micropathways (transdermal area), and then into blood circulation.

Patch Manufacturing. A patch was manufactured based on dispensing or tableting procedure known by those skilled in the art. Individual formulations utilized different ingredients as described herein. The patch includes a backing, a matrix including a non-aggregating peptide and ingredients disposed within the matrix, and a release liner, wherein the release liner is configured to be removed before application to the subject's skin. The non-aggregating peptide is p3-Alcβ9-19. In the dispensing method, all ingredients were dissolved in water and ethanol mixed solvent. The predetermined amount of ingredient solvents was dispensed on the non-woven pad in a patch without a release liner. The dispensed patch was moved to dry chamber at 50° C. until drying. The dried patch was removed from the dry chamber and stayed at room temperature for 30 minutes. The patch was covered by a release liner, then cut into the designated size. The individual patch was packed with a desiccant in the aluminum laminated pouch. In the tableting method, all ingredients were mixed and weighted as a designated amount. The weighed powder was transferred into the designated size of die punch for the compression machine. The die was set to the machine and compressed. The thin rectangular tablet was taken and placed on the non-woven pad in a patch without a release liner. The patch was covered by a release liner, then cut into the designated size. The individual patch was packed with a desiccant in the aluminum laminated pouch.

### Example 1. Administration Routs of p3-Alcβ

Nonparental Administration Routes. p3-Alcβ9-19 (5 mg/head) was administered in oral, nasal, or passive transdermal routes in rates. No absorption was observed in all administrations. p3-Alcβ9-19 is a hydrophilic peptide, and its molecular weight is 1256 Dalton. These physicochemical properties may influence poor membrane permeability through gastrointestinal, nasal, and stratum corneum.

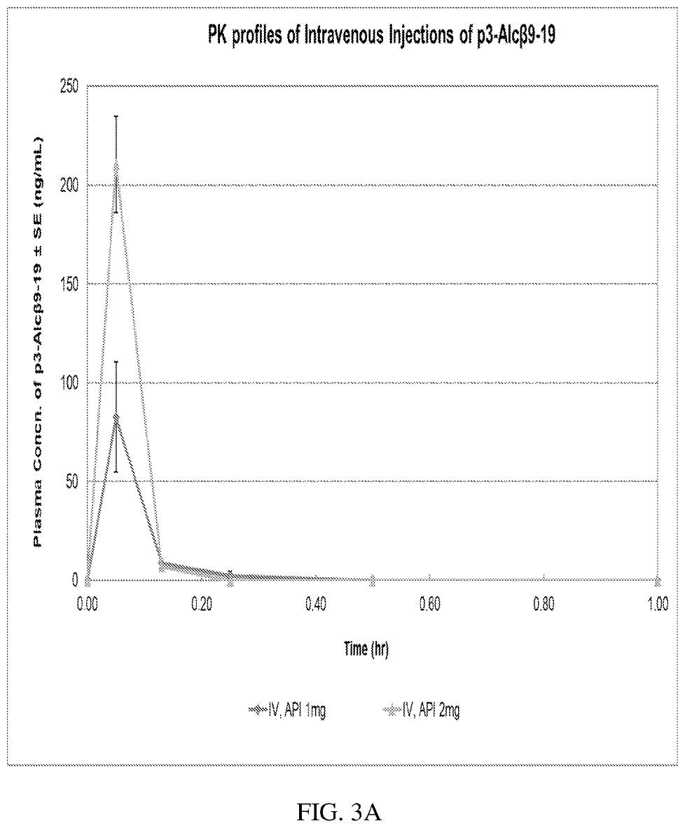

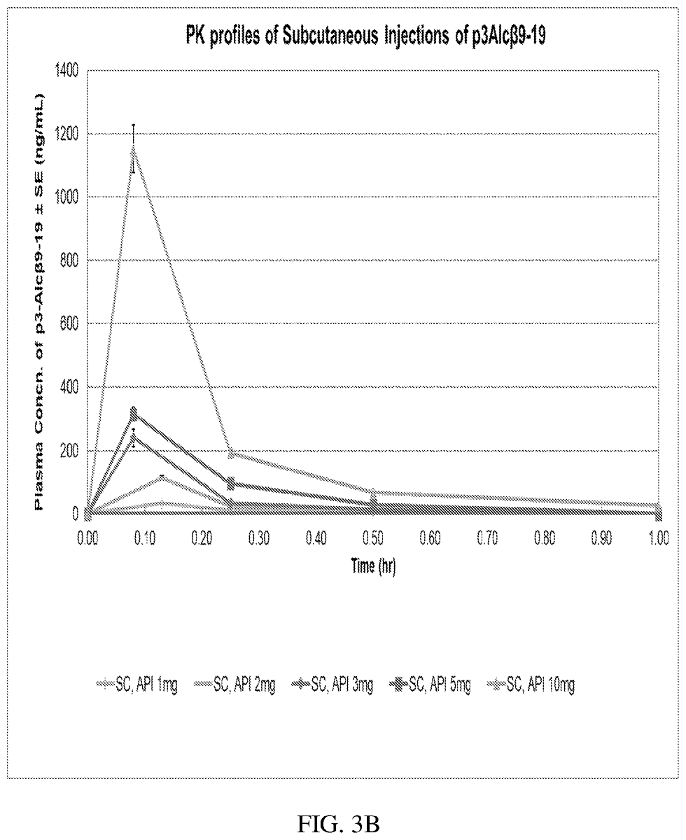

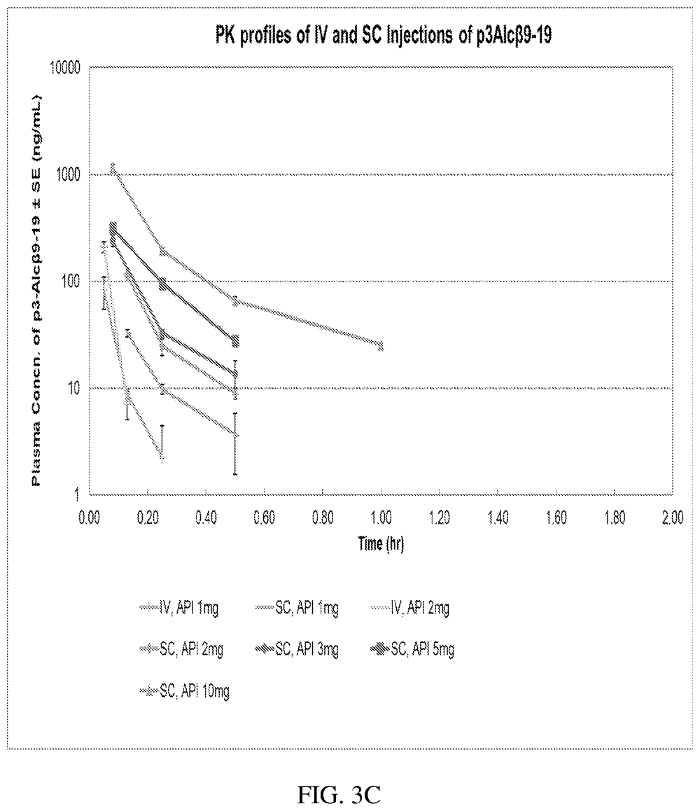

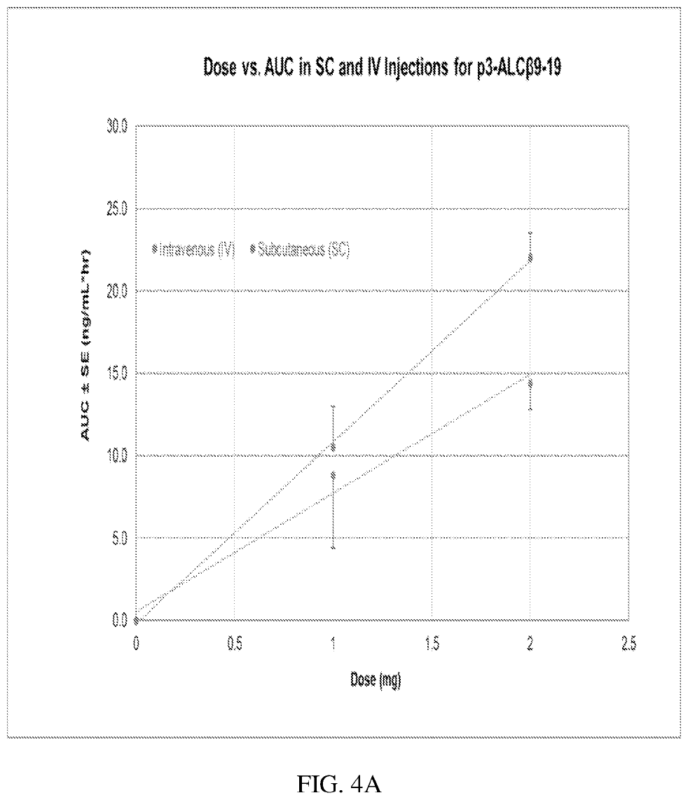

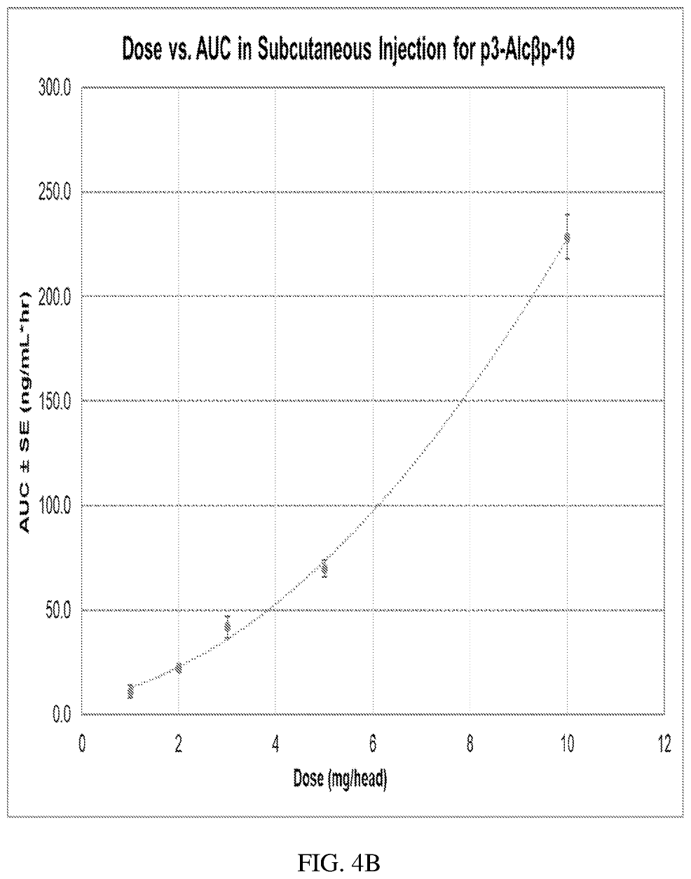

Parenteral Administration Routes. FIGS. 3A, 3B and 3C illustrate PK profiles of p3-Alcβ9-19 after intravenous and subcutaneous administration in rats (mean±SE, n=4). FIG. 3A illustrates a line graph depicting PK profiles of p3-Alcβ9-19 after intravenous administration in rats (1 and 2 mg/body); FIG. 3B illustrates a line graph depicting PK profiles of p3-Alcβ9-19 after subcutaneous administration (1 to 10 mg/body); FIG. 3C illustrates a logarithmic scale graph depicting PK profiles of p3-Alcβ9-19 in logarithmic scale. FIG. 4A illustrates a line graph depicting Dose responses of p3-Alcβ9-19 after intravenous and subcutaneous administration in rates; FIG. 4B illustrates a line graph depicting Dose responses of p3-Alcβ9-19 after subcutaneous administration in rats (Mean±SE, n=4).

The PK profiles of intravenous (IV) and subcutaneous (SC) injections showed rapid absorption and quick elimination in rats. The blood concentrations in SC were slightly lasted than IV, but it disappeared within 1 hours. The dose dependent increase was observed in both IV and SC injections. The bioavailability in SC may be similar or slightly higher than IV injection.

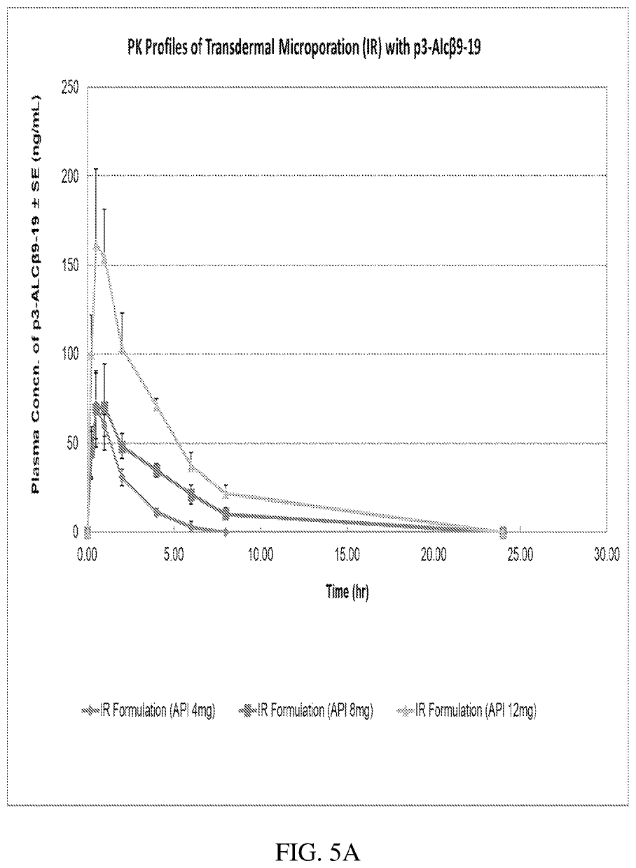

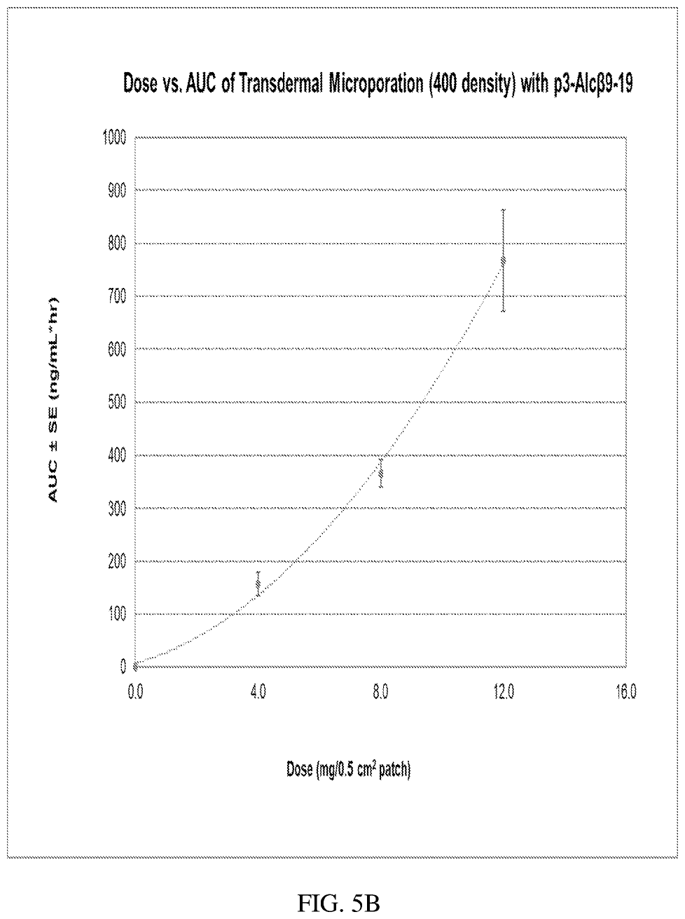

p3-Alcβ Delivery using Transdermal Microporation in Rats. FIG. 5A illustrates a line graph depicting immediate release (IR) formulations. FIG. 5B illustrates a line graph depicting dose response from transdermal microporation (400 density, 4 mJ/filament). Tables 3 and 4 describe the formulations and results of this study, respectively.

TABLE 3

| Ingredients | AARC009R-G1 | | AARC009R-G2 | | AARCO09R-G3 | |
|---|---|---|---|---|---|---|
| p3-Alcβ9-19 | 4 | mg | 8 | mg | 12 | mg |
| Sucrose | 0.5 | mg | 0.5 | mg | 0.5 | mg |
| Methylparaben | 0.2 | mg | 0.2 | mg | 0.2 | mg |
| Propylparaben | 0.02 | mg | 0.02 | mg | 0.02 | mg |

*Transdermal area: 0.5 cm²

TABLE 4

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| AARC009R-G1 | 156.9 | 22.0 | 545.1 | 76.4 | 78.7 | 12.2 | 0.8 | 0.1 |
| AARC009R-G2 | 365.8 | 26.2 | 635.3 | 45.6 | 73.5 | 23.1 | 0.9 | 0.1 |
| AARC009R-G3 | 767.8 | 95.5 | 889.1 | 110.6 | 187.8 | 39.5 | 0.9 | 0.1 |

*aBA (absolute bioavailability) vs IV (2 mg/body)

p3-Alcβ9-19 delivery using transdermal microporation showed higher aBA between 545 and 889% against IV (2 mg/body). The dose dependent increase was observed transdermal microporation system. The microporation delivery was the highest even compared to IV and SC injections. It suggested parenteral routes may be affected by in vivo stability of p3-Alcβ9-19 and the microporation delivery may avoid a first-pass effect (metabolism) and sustained release of p3-Alcβ9-19 rather than injections showed higher bioavailability.

### Example 2. Brain Delivery of p3-Alcβ Using Transdermal Microporation in Mice

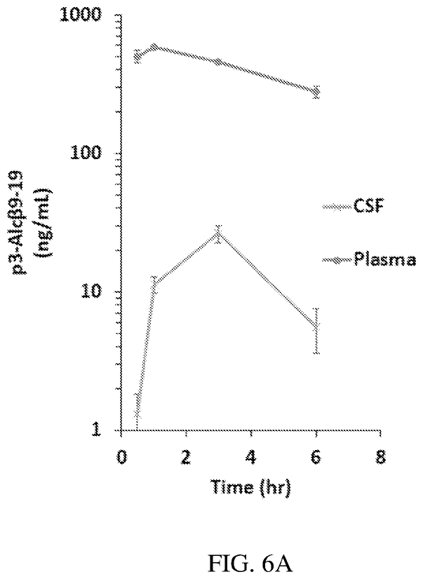

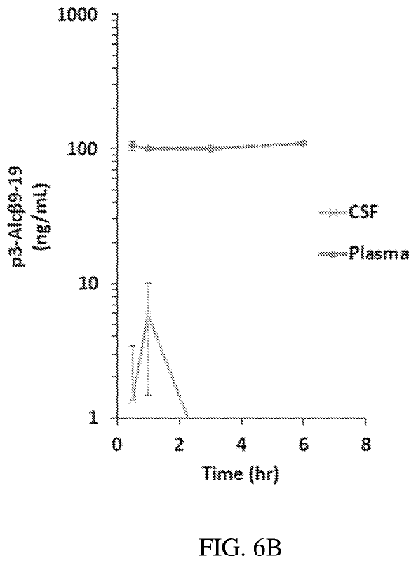

The formulation used for the study is provided in Table 5. FIG. 6A illustrates a logarithmic scale graph depicting changes in blood and central concentration after IR formulation containing p3-Alcβ9-19 using transdermal microporation in mice. FIG. 6B illustrates a logarithmic scale graph depicting changes in blood and central concentration after sustained release (SR) formulation containing p3-Alcβ9-19 using transdermal microporation in mice. Table 6 describes PK parameters in plasma after administrations of p3-Alcβ9-19 in mice.

TABLE 5

| Ingredients | IR formulation* | | SR formulation* | |
|---|---|---|---|---|
| p3-Alcβ9-19 | 1 | mg | 1 | mg |
| Lactose Monohydrate | 1 | mg | 1 | mg |
| Disodium Citrate Sesquihydrate | — | | 2.23 | mg |
| Methylparaben | 0.05 | mg | 0.05 | mg |
| Propylparaben | 0.005 | mg | 0.005 | mg |

*Transdermal area: 0.5 cm²

TABLE 6

| | | Cmax | Tmax | AUC₀₋₃₆₀ | BA |
|---|---|---|---|---|---|
| Group | Dose | (ng/mL) | (min) | (ng/mL min) | (%) |
| Transdermal | 1000 μg | 587.3 ± 7.1 | 60 | 152368.3 ± 9115.1 | 774 |
| Microporation | | | | | |
| (IR formulation)** | | | | | |
| Transdermal | 1000 μg | 110.8 ± 3.6 | — | 35748.9 ± 712.4 | 182 |
| Microporation | | | | | |
| (SR formulation)*** | | | | | |
| Subcutaneous Injection | 30 μg | 25.5 ± 12.5 | 18.8 ± 7.5 | 590.6 ± 322.2 | 100 |

*BA (relative bioavailability) vs SC (30 μg/body)
**Transdermal microporation: 400 density, 3 mJ/filament
***Transdermal microporation: 100 density, 2 mJ/filament

Table 7 describes PK parameters in cerebrospinal fluid (CSF) after administrations of p3-Alcβ9-19 in mice.

TABLE 7

| | | Cmax | Tmax | AUC₀₋₃₆₀ | BA |
|---|---|---|---|---|---|
| Group | Dose | (ng/mL) | (min) | (ng/mL min) | (%) |
| Transdermal | 1000 μg | 26.3 ± 7.5 | 180 | 5335.4 ± 664.9 | 266 |
| Microporation | | | | | |
| (IR formulation) | | | | | |
| Transdermal | 1000 μg | 5.79 ± 4.3 | 60 | 530.15 ± 61.4 | 26 |
| Microporation | | | | | |
| (SR formulation) | | | | | |
| Subcutaneous Injection | 30 μg | 14.9 ± 10.7 | 15.0 ± 5.8 | 300.9 ± 64.7 | 100 |

*BA (relative bioavailability) vs SC (30 μg/body)
**Transdermal microporation: 400 density, 3 mJ/filament
***Transdermal microporation: 100 density, 2 mJ/filament

FIG. 6A and FIG. 6B shows the changes of p3-Alcβ9-19 concentration in plasma and cerebrospinal fluid (CSF) when IR formulation (FIG. 5A) or SR formulation (FIG. 5B) containing p3-Alcβ9-19 was administered to mice using a transdermal microporation device. Tables 2 and 3 show when IR formulation containing 1 mg of p3-Alcβ9-19 was administered to mice, the maximum blood concentration (Cmax) was about 600 ng/mL at 1 hour after administration, and the blood concentration was maintained at about 300 ng/mL for up to 6 hours. In CSF, >20 ng/mL was shown 3 hours after administration, and a concentration of about 5 ng/mL was detected 6 hours later. It showed good central transfer that the transfer rate of p3-Alcβ9-19 from blood to CSF was about 3.5% (BA in CSF/BA in plasma×100%). SF formulation showed a sustained plasma concentration in mice, and CSF concentration was close to the detection limit.

### Example 3. Pharmacological Effects of p3-Ales Transdermal Microporation in Monkey

In this example, two patch formulations of the disclosure are exemplified. It suggested the use of an IR patch is considered preferable to achieve effective elevation of mitochondrial activity.

The mitochondrial activation effect of p3-Alcβ9-19 transdermal microporation was investigated in Rhesus monkeys. The formulation used for the study showed below in Table 8. As a result, [18F]BCPP-EF accumulation, that is, activation of mitochondria, was observed by PET measurement 1.5 hours after administration of p3-Alcβ9-19 using transdermal microporation. In addition, activation continued in some areas even after 6.5 hours.

TABLE 8

| Ingredients | IR formulation* | | SR formulation* | |
|---|---|---|---|---|
| p3-Alcβ9-19 | 5 | mg | 5 | mg |
| Lactose Monohydrate | 1 | mg | 1 | mg |
| Disodium Citrate | — | | 4 | mg |
| Methylparaben | 0.1 | mg | 0.1 | mg |
| Propylparaben | 0.01 | mg | 0.01 | mg |

*Transdermal area: 0.5 cm²

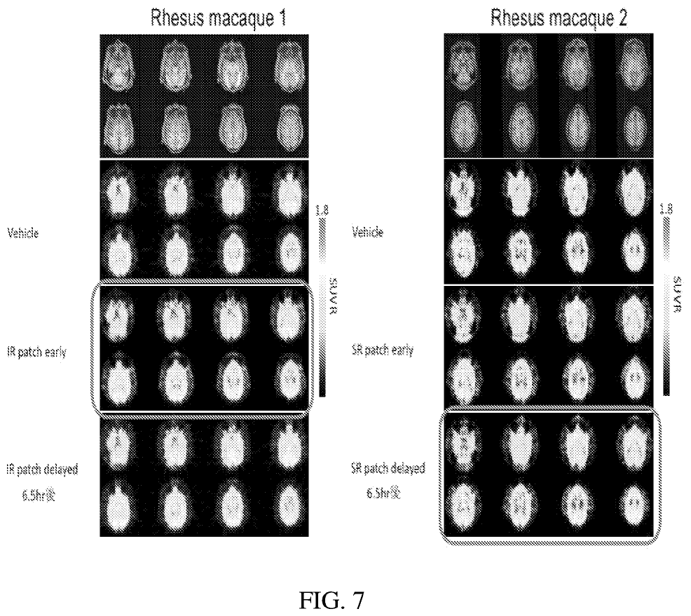

FIG. 7 illustrates two images depicting changes in mitochondrial activity in the brain after IR formulation (left) and SR formulation (right) containing p3-Alcβ9-19 using transdermal microporation in monkeys. Transdermal microporation was applied 400 density and 4 mJ/filament for IR formulation, 100 density and 3 mJ/filament for SR formulation. Increases in mitochondrial activity are found in monkeys (marked by red rectangles) with immediate response (early) at one hour after administration of IR formulation containing p3-Alcβ9-19 and slow response (delay) at 6.5 hours after its administration with SR formulation containing p3-Alcβ9-19 against control (vehicle).

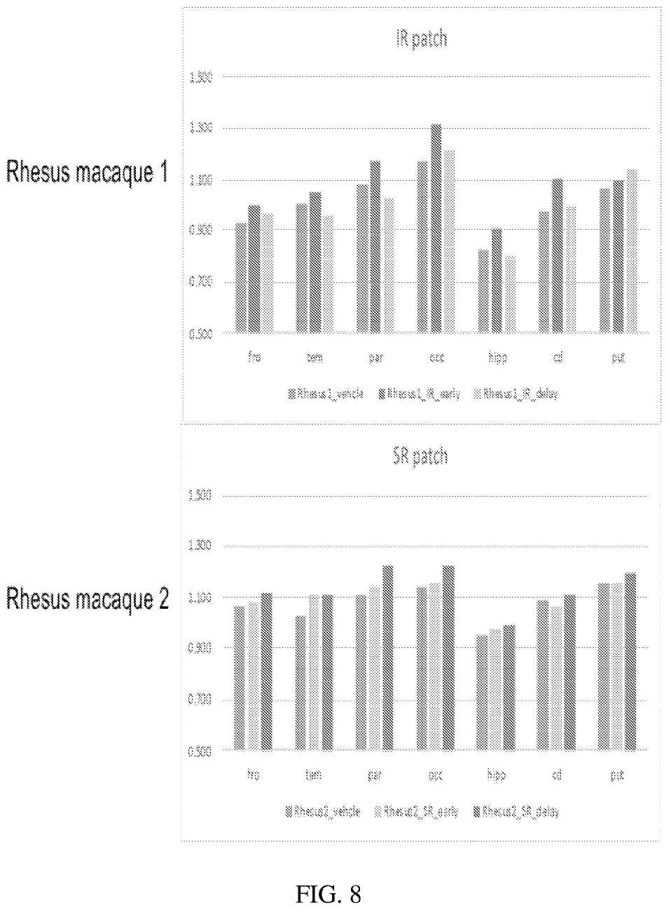

FIG. 8 illustrates two bar graphs describing the intensity from FIG. 7. In FIG. 8, the following abbreviations are used: fro: Frontal Lobes, tem: Temporal Lobes, par: Parietal Lobes, occ: Occipital Lobes, hipp: Hippocampus, cd: Caudate, put: Putamen. FIG. 8 (upper) was applied IR formulation and FIG. 8 (bottom) applied SR formulation. In FIG. 8 (upper), intensities at one hour showed higher at all regions compared to control (vehicle), but it decreased at 6.5 hours at all regions except Putamen. In FIG. 8 (bottom), intensities gradually increased in all regions.

FIG. 9 illustrates a bar graph depicting the percentage increase in mitochondrial activity in various regions of the brain at one hour after administration of IR formulation containing p3-Alcβ9-19 (5 or 10 mg/body equivalent to 0.5 or 1.0 mg/kg, respectively). The percentage increase was raised at 10 mg/body than 5 mg/body except at Frontal Lobes and Putamen regions.

### Example 4. Control of PK Profile Using IR Formulation

Table 9 describes IR formulations used for the study that included p3-Alcβ9-19, enhancer (i.e. non-reducing sugar: Sucrose), anti-microbial agent (combination of methylparaben and propylparaben or benzalkonium chloride). The transdermal microporation was applied with different combination of density and energy level to investigate of PK modifications.

TABLE 9

| Ingredients | AALC004R | | AALC005R | | AALC006R | | AALC029R | | AALCO30R | |
|---|---|---|---|---|---|---|---|---|---|---|
| p3-Alcβ9-19 | 4 | mg | 4 | mg | 8 | mg | 5 | mg | 5 | mg |
| Sucrose | 0.5 | mg | 0.5 | mg | 1 | mg | 1 | mg | 0.5 | mg |

| | | | | | | | | |
|---|---|---|---|---|---|---|---|---|
| Methylparaben | 0.2 | mg | 0.2 | mg | 0.2 | mg | — | — |
| Propylparaben | 0.02 | mg | 0.02 | mg | 0.02 | mg | — | — |

| | | | | | | | |
|---|---|---|---|---|---|---|---|
| Benzalkonium Chloride | — | — | — | 0.025 | mg | 0.025 | mg |

*Transdermal area: 0.5 cm²

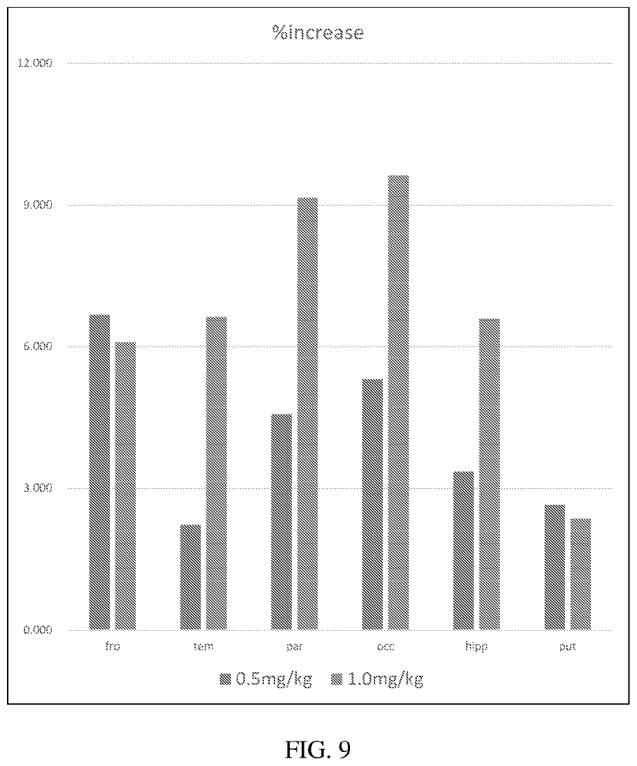

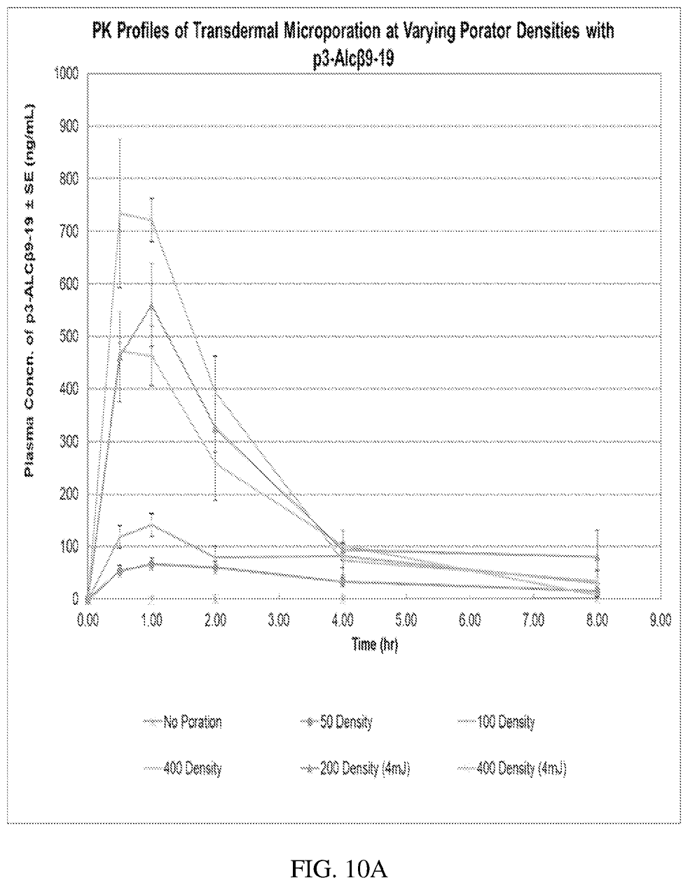

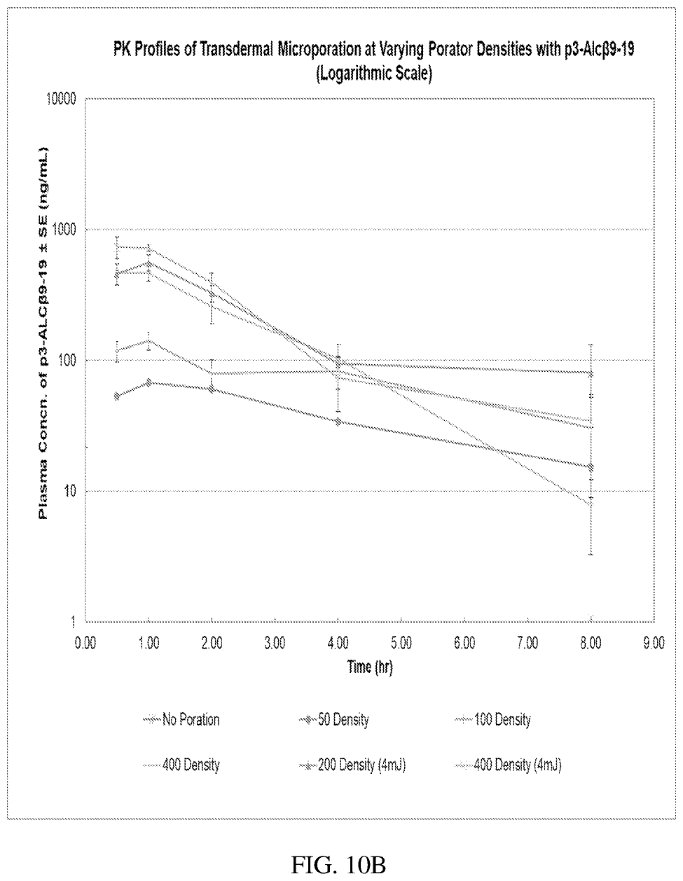

FIG. 10A illustrates a line graph depicting the PK profiles using non-reducing sugar formulations and p3-Alcβ 3 concentration over time with different densities at 4 mj/filament; FIG. 10B illustrates a logarithmic scale graph depicting the p3-Alcβ concentration over time.

Table 10 shows the results of types of PK profiles using IR formulations containing p3-Alcβ9-19 with different microporation conditions, including the results of FIG. 10A. In immediate release (IR) type formulations, PK profiles may be controlled by changing transdermal microporation conditions. No absorption was observed without transdermal microporation. The 200 density at 4 mJ/filament and 400 density between 2 and 4 mJ/filament showed immediate PK profiles for p3-Alco. On the other hand, microporation conditions (50≤Density≤200 density and <4 mJ/filament) provided a sustained PK profile for p3-Alcβ.

TABLE 10

| | 2 mJ/filament | 3 mJ/filament | 4 mJ/filament |
|---|---|---|---|
| 50 density | — | — | Sustained PK profile |
| 100 density | Sustained PK profile | — | Sustained PK profile |
| 200 density | Sustained PK profile | Sustained PK profile | Immediate PK profile |
| 400 density | Immediate PK profile | — | Immediate PK profile |

### Example 5. Control of PK Profile Using SR Formulation

Table 11 describes SR formulations used for the study, including p3-Alcβ9-19, enhancer (i.e. non-reducing sugar: Sucrose), PK modifier (Disodium Citrate Sesquihydrate), anti-microbial agent (Methylparaben/propylparaben).

TABLE 11

| Ingredients | AALC011R-G4 | | AALC011R-G5 | | AALC011R-G6 | |
|---|---|---|---|---|---|---|
| p3-Alcβ9-19 | 8 | mg | 8 | mg | 8 | mg |
| Sucrose | 0.5 | mg | 0.5 | mg | 0.5 | mg |
| Disodium Citrate Sesquihydrate | 1 | mg | 2 | mg | 4 | mg |
| Methylparaben | 0.2 | mg | 0.2 | mg | 0.2 | mg |
| Propylparaben | 0.02 | mg | 0.02 | mg | 0.02 | mg |

*Transdermal area: 0.5 cm²

Table 12 provides a summary of types of PK profiles using SR formulations containing p3-Alcβ9-19 with different content of PK modifiers. The PK modifier (i.e. containing organic acid, its salt, or a combination thereof) is added in sustained release (SR) type formulations. The sustained PK profile for p3-Alcβ9-19 was obtained with 4 mg of disodium citrate sesquihydrate at 200 density, 4 mJ/filament.

TABLE 12

| | AALC011R-G4 | AALC011R-G5 | AALC011R-G6 |
|---|---|---|---|
| 200d, 4 mJ | Immediate PK profile | Immediate PK profile | Sustained PK profile |

Table 13 describes SR formulations used for the study included p3-Alcβ9-19, enhancer (non-reducing sugar: Sucrose), PK modifier (Disodium Citrate Sesquihydrate), anti-microbial agent (Methylparaben/propylparaben). The transdermal microporation was applied with a combination of different densities and energy levels using a PK modifier (4 mg of disodium citrate sesquihydrate).

TABLE 13

| | | | AALC013R and | |
|---|---|---|---|---|
| Ingredients | AALCO07R | | AALC014R | |
| p3-Alcβ9-19 | 8 | mg | 8 | mg |
| Sucrose | 1 | mg | 0.5 | mg |
| Disodium Citrate Sesquihydrate | 4 | mg | 4 | mg |
| Methylparaben | 0.2 | mg | 0.2 | mg |
| Propylparaben | 0.02 | mg | 0.02 | mg |

*Transdermal area: 0.5 cm²

Table 14 summarizes PK profiles using different microporation conditions with PK modifier. The sustained release (SR) formulations containing disodium citrate sesquihydrate showed sustained PK profiles with 4 mg of disodium citrate sesquihydrate at lower than 400 density and 4 mJ/filament.

TABLE 14

| | 2 mJ | 3 mJ | 4 mJ |
|---|---|---|---|
| 100 density | Sustained PK profile | — | Sustained PK profile |
| 200 density | Sustained PK profile | — | Sustained PK profile |
| 400 density | — | — | Sustained PK profile |

Table 15 summarizes PK profile control using IR and SR formulation types containing p3-Alcβ using transdermal microporation. The immediate PK profile is provided with IR type formulation (without PK modifier) at microporation conditions of less than 200 density and 2-4 mJ/filament. The sustained PK profiles may be obtained with IR formulation at 50≤Density≤200 density, and <4 mJ/filament and SR formulations containing PK modifier at less than 400 density and 4 mJ/filament.

TABLE 15

| | Immediate PK Profile | Sustained PK Profile |
|---|---|---|
| IR type formulations | ≥200 density | 50 ≤ Density ≤ 200 |
| (without organic acids) | and ≥2~4 | density, and <4 |
| | mJ/filament | mJ/filament |
| SR type formulations | — | ≤400 density and ≤4 |
| (with organic acids) | | mJ/filament PK modifier |
| | | (i.e. Citric Acid: more |
| | | than 2 mg, Disodium |
| | | Citrate: more than 4 mg) |

### Example 6. Enhancement of p3-Alcβ Delivery with Enhancers

Table 16 describes dry patch formulations to investigate on the effects of enhancers (sugars) on the delivery of p3-Alcβ9-19 using transdermal microporation. The enhancers were selected from non-reducing sugar (sucrose, trehalose, D-mannitol and D-sorbitol) and reducing sugars (lactose and maltose).

TABLE 16

| Ingredients | AALC021R-G1 | AALC021R-G2 | AALC021R-G3 | AALC021R-G4 | AALC021R-G5 | AALC021R-G6 |
|---|---|---|---|---|---|---|
| p3-Alcβ9-19 | 5 mg | 5 mg | 5 mg | 5 mg | 5 mg | 5 mg |
| Enhancer | Sucrose | Lactose | Trehalose | Maltose | D-Mannitol | D-Sorbitol |
| | 1 mg | 1 mg | 1 mg | 1 mg | 1 mg | 1 mg |
| Methyl | 0.1 mg | 0.1 mg | 0.1 mg | 0.1 mg | 0.1 mg | 0.1 mg |
| paraben | | | | | | |
| Propyl | 0.01 mg | 0.01 mg | 0.01 mg | 0.01 mg | 0.01 mg | 0.01 mg |
| paraben | | | | | | |

*Transdermal area: 0.5 cm²

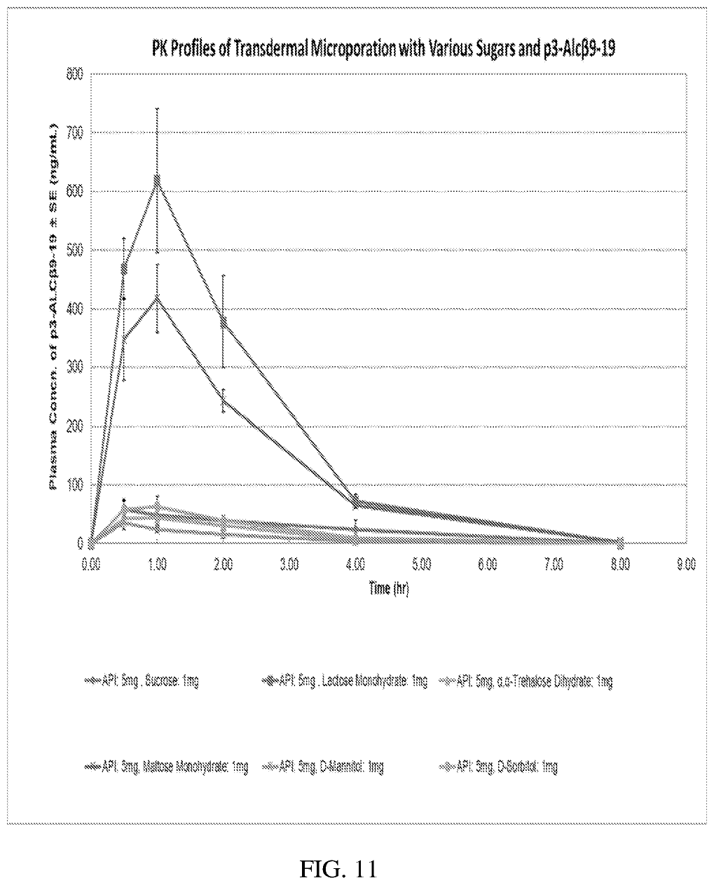

FIG. 11 illustrates PK profiles with non-reducing or reducing sugars. The reducing sugars (lactose and maltose) showed a higher delivery than non-reducing sugars. Table 17 describes the results from FIG. 11. All non-reducing or reducing sugars enhanced p3-Alcβ absorption compared to IV injections. The aBA for non-reducing sugars was between 194.3 and 547.3%. Especially, reducing sugars such as maltose and lactose showed remarkable enhancement for p3-Alcβ9-19 using transdermal microporation (aBA: 2923.3 and 4121.7%).

TABLE 17

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| AALC021R-G1** | 196.9 | 41.7 | 547.3 | 116.0 | 78.9 | 13.3 | 1.9 | 0.8 |
| AALC021R-G2** | 1483.0 | 224.4 | 4121.7 | 623.5 | 618.2 | 122.1 | 1.0 | 0.0 |
| AALC021R-G3** | 118.2 | 10.9 | 328.6 | 30.3 | 50.4 | 7.1 | 0.9 | 0.1 |
| AALC021R-G4** | 1051.8 | 85.7 | 2923.3 | 238.2 | 426.3 | 58.3 | 0.9 | 0.1 |
| AALC021R-G5** | 69.9 | 10.1 | 194.3 | 28.1 | 40.3 | 8.7 | 0.9 | 0.4 |
| AALC021R-G6** | 163.1 | 21.5 | 452.8 | 59.8 | 66.8 | 14.5 | 0.8 | 0.1 |

*aBA (absolute bioavailability) vs IV (2 mg/body)
**Transdermal microporation: 400 density, 4 mJ/filament

### Example 7. Effects of Anti-Microbial Aments on p3-Alcβ Delivery

Anti-microbial agents are added as preservatives or anti-microbial effectiveness to avoid a risk of infections. Table 18 describes examples of formulations with anti-microbial agents.

TABLE 18

| Ingredients | AALC0028R-G1 | AALC0028R-G2 | AALC0028R-G3 | AALC0028R-G4 |
|---|---|---|---|---|
| p3-Alcβ9-19 | 10 | 10 | 10 | 10 |
| Lactose | 2 | 2 | — | — |
| Monohydrate | | | | |
| Maltose | — | — | 2 | 2 |
| Methylparaben | — | 0.15 | — | 0.15 |
| Propylparaben | — | 0.015 | — | 0.015 |
| Benzalkonium | 0.025 | — | 0.025 | — |
| Chloride | | | | |

*Transdermal area: 0.5 cm²

Table 19 describes the PK parameter after applications of the formulations using transdermal microporation (400 density, 4 mJ/filament) in rats. Suggesting that reducing sugars worked with anti-microbial agents (a combination of methylparaben and propylparaben, or bezalkonium chloride formulation).

TABLE 19

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| AALC0028R-G1 | 1817.4 | 211.5 | 2536.6 | 295.2 | 700.2 | 250.1 | 0.6 | 0.1 |
| AALC0028R-G2 | 1198.5 | 46.2 | 1672.8 | 64.4 | 343.5 | 46.3 | 1.1 | 0.3 |
| AALC0028R-G3 | 1402.6 | 320.0 | 1957.6 | 446.7 | 425.1 | 83.2 | 0.9 | 0.1 |
| AALC0028R-G4 | 1452.2 | 159.7 | 2026.8 | 222.9 | 578.3 | 69.3 | 0.7 | 0.2 |

*aBA (absolute bioavailability) vs IV (2 mg/body)
**Transdermal microporation: 400 density, 4 mJ/filament

### Example 8. Immediate and Sustained PK Delivery Using Reducing Sugar Formulations

Table 20 describes IR (without PK modifier) and SR (with PK modifier) formulations containing reducing sugar (lactose) and p3-Alcβ9-19 to control PK profiles.

TABLE 20

| | IR formulation | | | | SR formulation | | | |
|---|---|---|---|---|---|---|---|---|
| Ingredient | G1 | | G2 | | G3 | | G4 | |
| p3-Alcβ9-19 | 5 | mg | 5 | mg | 5 | mg | 5 | mg |
| Sucrose | 0.5 | mg | — | | 1 | mg | — | |
| Lactose Monohydrate | — | | 0.5 | mg | — | | 2 | mg |
| Disodium Citrate | — | | — | | 4 | mg | 4 | mg |
| Sesquihydrate | | | | | | | | |
| Methylparaben | 0.2 | mg | 0.2 | mg | 0.2 | mg | 0.2 | mg |
| Propylparaben | 0.02 | mg | 0.02 | mg | 0.02 | mg | 0.02 | mg |

*Transdermal area: 0.5 cm²

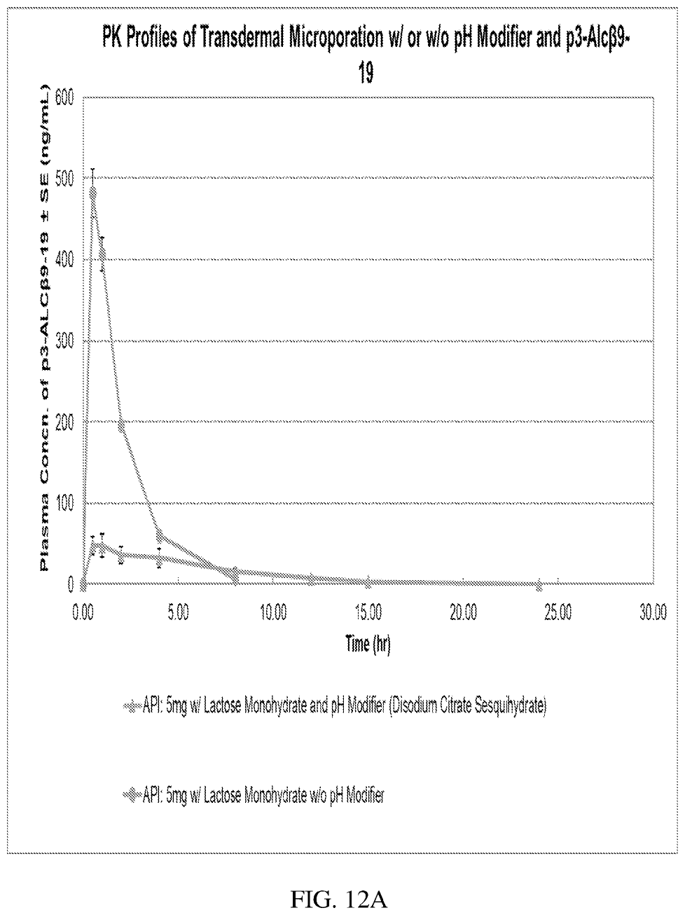

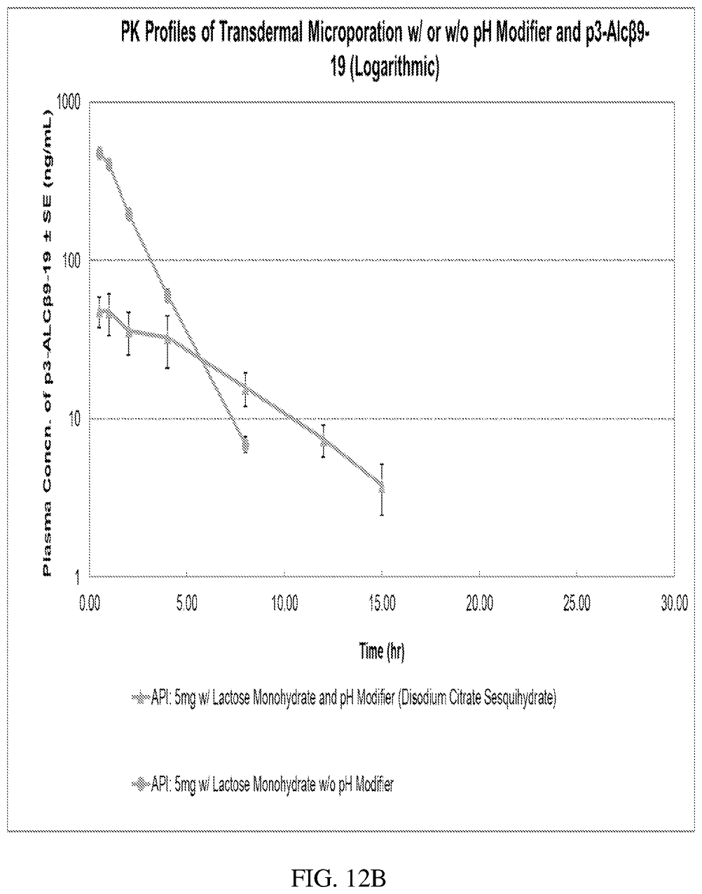

FIG. 12A illustrates an example of the line graph depicting the PK profiles of IR and SR formulation containing a reducing sugar (lactose) and p3-Alcβ 3 formulations in rats (G2 and G4); FIG. 12B illustrates a logarithmic scale graph depicting the p3-Alcβ concentration over time. After applications of JR and SR formulations containing a reducing sugar using transdermal microporation, the plasma concentration of p3-Alcβ9-19 showed typical immediate and sustained PK profiles.

Table 21 describes the results of PK parameters after applications of the formulations using transdermal microporation in rats. IR and SR formulations containing reducing sugar showed a higher BA compared to non-reducing sugar formulations, respectively.

TABLE 21

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| G1** | 208.0 | 16.9 | 578.0 | 46.9 | 72.0 | 12.7 | 1.0 | 0.0 |
| G2** | 1036.4 | 10.7 | 2880.5 | 29.6 | 481.9 | 29.6 | 0.5 | 0.0 |
| G3*** | 210.3 | 51.9 | 584.6 | 144.4 | 31.5 | 9.2 | 1.9 | 0.8 |
| G4*** | 323.5 | 74.7 | 899.2 | 207.5 | 51.1 | 13.1 | 0.6 | 0.1 |

*aBA (absolute bioavailability) vs IV (2 mg/body)
**Transdermal microporation: 400 density, 4 mJ/filament
***Transdermal microporation: 100 density, 3 mJ/filament

### Example 9. Effects of Density on p3-Ales Delivery Using Reducing Sumar Formulations

Table 22 describes a formulation to determine p3-Alcβ9-19 delivery with different microporation densities.

TABLE 22

| Ingredients | AALC007R | |
|---|---|---|
| p3-Alcβ9-19 | 5 | mg |
| Lactose Monohydrate | 1 | mg |
| Benzalkonium Chloride | 0.025 | mg |

*Transdermal area: 0.5 cm²

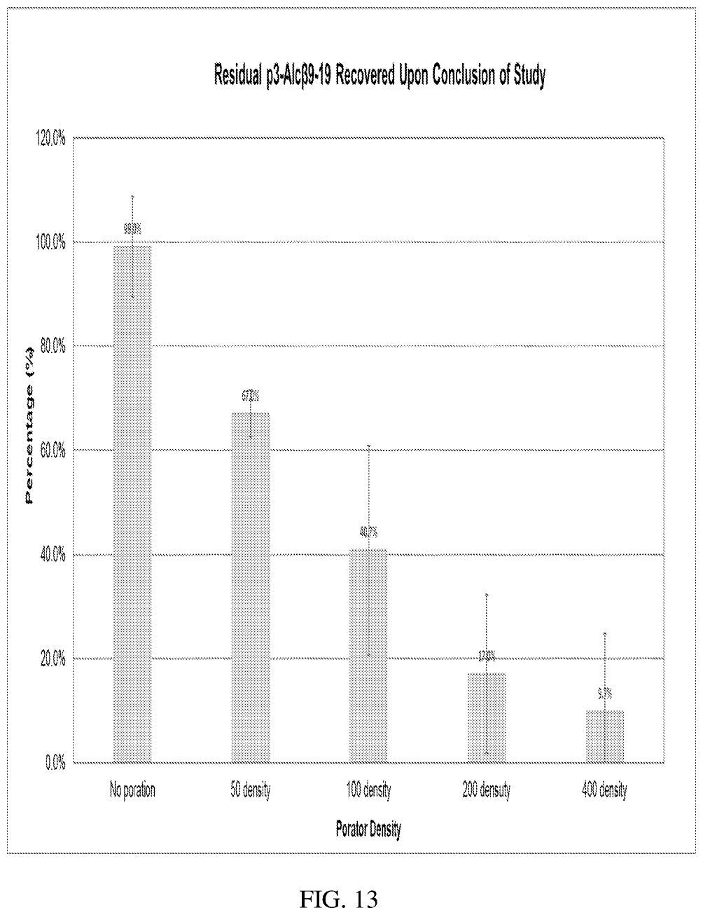

FIG. 13 illustrates the drug residual at 24 hours after an application of reducing sugar formulations using transdermal microporation in rats (4 mJ/filament). p3-Alcβ9-19 was recovered about 100% without microporation. The drug recovery was decreased as increasing of transdermal microporation densities. 10% to 20% of p3-Alcβ9-19 was recovered at 200 to 400 density. More than 200 density shows effective delivery of p3-Alcβ9-19.

### Example 10. Optimization of Enhancer Content in Reducing Sugar Formulations

Table 23 describes formulations used for the investigation on the effect of enhancer content in p3-Alcβ transdermal microporation.

TABLE 23

| | AALC0019R | | | | | | AALC0020R | | | | | | AALC0034R | |
|---|---|---|---|---|---|---|---|---|---|---|---|---|---|---|
| Ingredients | G1 | | G2 | | G3 | | G4 | | G5 | | G6 | | G7 | |
| p3-Alcβ9-19 | 5 | mg | 5 | mg | 5 | mg | 5 | mg | 5 | mg | 10 | mg | 5 | mg |
| Lactose | 0.1 | mg | 0.25 | mg | 0.5 | mg | 1 | mg | 2 | mg | 0.5 | mg | — | |
| Monohydrate | | | | | | | | | | | | | | |
| Methylparaben | 0.05 | mg | 0.05 | mg | 0.05 | mg | 0.05 | mg | 0.05 | mg | 0.1 | mg | 0.05 | mg |
| Propylparaben | 0.005 | mg | 0.005 | mg | 0.005 | mg | 0.005 | mg | 0.005 | mg | 0.01 | mg | 0.005 | mg |
| Pad size | 0.25 | cm² | 0.25 | cm² | 0.25 | cm² | 0.25 | cm² | 0.25 | cm² | 0.5 | cm² | 0.25 | cm² |
| (Transdermal | | | | | | | | | | | | | | |
| area) | | | | | | | | | | | | | | |
| Ratio | 50:1 | | 25:1 | | 10:1 | | 5:1 | | 2.5:1 | | 20:1 | | — | |
| p3-Alcβ9- | | | | | | | | | | | | | | |
| 19:Lactose | | | | | | | | | | | | | | |

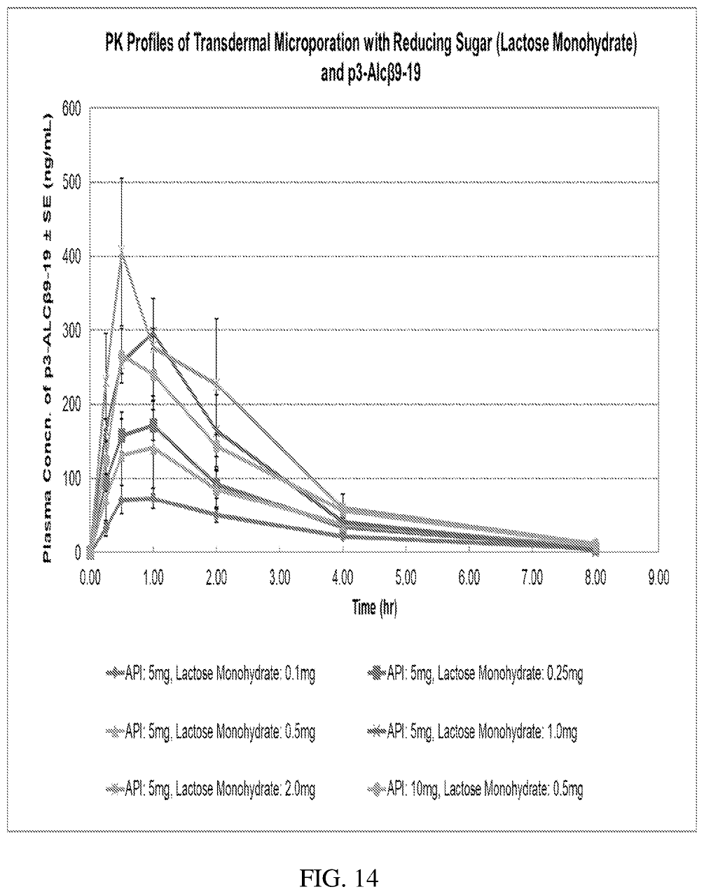

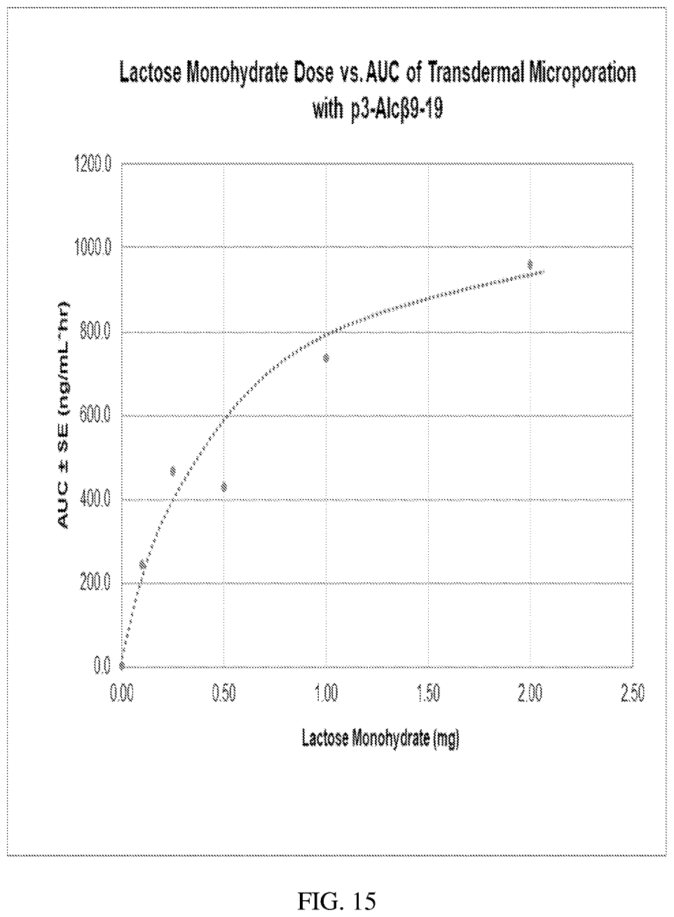

FIG. 14 illustrates a line graph depicting rats' PK profiles with different enhancer content (reducing sugar, lactose). Table 24 describes the PK parameters from FIG. 14. FIG. 15 illustrates a line graph comparing the AUC versus lactose content.

TABLE 24

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| G1** | 243.4 | 38.8 | 676.5 | 107.9 | 77.2 | 16.6 | 0.8 | 0.1 |
| G2** | 466.9 | 55.0 | 1297.7 | 152.9 | 175.8 | 20.0 | 0.8 | 0.1 |
| G3** | 430.2 | 153.0 | 1195.7 | 425.3 | 145.3 | 61.0 | 1.3 | 0.3 |
| G4** | 736.1 | 138.2 | 2045.7 | 384.0 | 296.9 | 6.1 | 1.0 | 0.0 |
| G5** | 958.7 | 243.7 | 2664.4 | 677.3 | 419.5 | 93.0 | 0.6 | 0.1 |
| G6** | 721.6 | 82.3 | 1002.7 | 114.4 | 269.6 | 38.4 | 0.6 | 0.1 |
| G7** | 3.4 | 3.3 | 9.5 | 9.3 | 6.7 | 6.7 | 0.6 | 0.5 |

*aBA (absolute bioavailability) vs IV (2 mg/body)
**Transdermal microporation: 400 density, 4 mJ/filament

The plasma concentration of p3-Alcβ9-19 was increased with increasing of enhancer content (G1-G5). No enhancement was observed without enhancer (G7). The ratio between p3-Alcβ and enhancer may have a relation to the enhancement factor.

### Example 11. Dose-Dependency of p3-Alcβ Delivery Using IR and SR Formulations

Immediate Delivery System. Tables 25 and 26 describe JR formulations containing p3-Alcβ3 to determine dose-dependency using transdermal microporation.

TABLE 25

| | AALC0024R | | | | | | | | | | AALC0026R | |
|---|---|---|---|---|---|---|---|---|---|---|---|---|
| Ingredients | G1 | | G2 | | G3 | | G4 | | G5 | | G1 | |
| p3-Alcβ9-19 | 3 | mg | 5 | mg | 10 | mg | 20 | mg | 25 | mg | 10 | mg |
| Lactose | 1 | mg | 1 | mg | 2 | mg | 4 | mg | 5 | mg | 2 | mg |
| Monohydrate | | | | | | | | | | | | |
| Methylparaben | 0.1 | mg | 0.1 | mg | 0.1 | mg | 0.2 | mg | 0.2 | mg | 0.2 | mg |
| Propylparaben | 0.01 | mg | 0.01 | mg | 0.01 | mg | 0.02 | mg | 0.02 | mg | 0.02 | mg |
| Pad size | 0.5 | cm² | 0.5 | cm² | 0.5 | cm² | 1 | cm² | 1 | cm² | 1 | cm² |
| (Transdermal Area) | | | | | | | | | | | | |

TABLE 26

| | AALC0022R | | | | AALC0032R | | | | | |
|---|---|---|---|---|---|---|---|---|---|---|
| Ingredients | G1 | | G2 | | G3 | | G4 | | G5 | |
| p3-Alcβ9-19 | 5 | mg | 10 | mg | 5 | mg | 20 | mg | 50 | mg |
| Lactose Monohydrate | 1 | mg | 2 | mg | 2 | mg | 4 | mg | 5 | mg |
| Methylparaben | 0.1 | mg | 0.1 | mg | 0.12 | mg | 0.48 | mg | 1.2 | mg |
| Propylparaben | 0.01 | mg | 0.01 | mg | 0.012 | mg | 0.048 | mg | 0.12 | mg |
| Pad size | 0.5 | cm² | 0.5 | cm² | 0.5 | cm² | 1 | cm² | 2 | cm² |
| (Transdermal Area) | | | | | | | | | | |

Table 27 shows PK parameters on immediate delivery with different p3-Alcβ9-19 dose using transdermal microporation in rats (400 density, 4 mJ/filament).

TABLE 27

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| AALC0024R-G1 | 1057.5 | 80.7 | 4247.1 | 324.2 | 504.3 | 42.6 | 0.75 | 0.14 |
| AALC0024R-G2 | 1364.2 | 440.7 | 3287.2 | 1061.9 | 598.1 | 228.9 | 1.00 | 0.00 |
| AALC0024R-G3 | 2267.9 | 265.8 | 2732.4 | 320.3 | 693.2 | 132.5 | 1.25 | 0.25 |
| AALC0024R-G4 | 4586.0 | — | 2762.6 | — | 1588.8 | — | 1.00 | — |
| AALC0024R-G5 | 2734.1 | — | 1317.6 | — | 1187.0 | — | 1.00 | — |
| AALC0026R-G1 | 2321.6 | 56.0 | 2797.1 | 67.5 | 923.7 | 43.19 | 0.75 | 0.17 |
| AALC0022R-G1 | 1308.8 | 283.4 | 3637.5 | 787.6 | 496.6 | 163.07 | 0.67 | 0.17 |
| AALC0022R-G2 | 1842.9 | 448.9 | 2560.9 | 623.8 | 593.0 | 166.57 | 0.83 | 0.17 |
| AALC0032R-G3 | 645.3 | 99.6 | 1801.3 | 277.9 | 204.6 | 49.55 | 1.13 | 0.31 |
| AALC0032R-G4 | 3127.0 | 600.5 | 2182.2 | 419.0 | 1033.1 | 319.84 | 0.88 | 0.13 |
| AALC0032R-G5 | 6633.9 | 1487.4 | 1851.8 | 415.2 | 2294.4 | 936.15 | 1.00 | 0.00 |

*aBA (absolute bioavailability) vs IV (2 mg/body)
**Transdermal microporation: 400 density, 4 mJ/filament

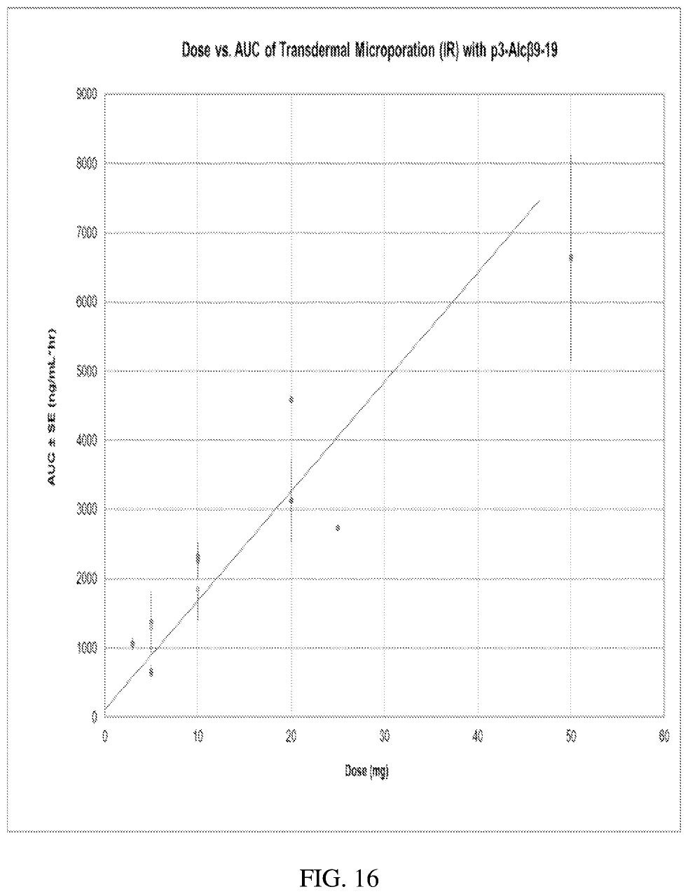

FIG. 16 illustrates the relationship between p3-Alcβ9-19 dose and AUC. The liner dose-dependency in immediate delivery was observed up to 50 mg of p3-Alcβ9-19 (tested dose).

Sustained Delivery System. Tables 28 and 29 describe SR formulations containing p3-Alcβ3 to determine dose-dependency using transdermal microporation.

TABLE 28

| | AALC0023R | | | | | | | | | | AALC0026R | |
|---|---|---|---|---|---|---|---|---|---|---|---|---|
| Ingredients | G1 | | G2 | | G3 | | G4 | | G5 | | G2 | |
| p3-Alcβ9-19 | 3 | mg | 5 | mg | 10 | mg | 20 | mg | 25 | mg | 10 | mg |
| Lactose Monohydrate | 1 | mg | 1 | mg | 2 | mg | 4 | mg | 5 | mg | 2 | mg |
| Disodium Citrate | 4.5 | mg | 4.5 | mg | 4.5 | mg | 8.9 | mg | 8.9 | mg | 8.9 | mg |
| Sesquihydrate | | | | | | | | | | | | |
| Methylparaben | 0.1 | mg | 0.1 | mg | 0.1 | mg | 0.2 | mg | 0.2 | mg | 0.2 | mg |
| Propylparaben | 0.01 | mg | 0.01 | mg | 0.01 | mg | 0.02 | mg | 0.02 | mg | 0.02 | mg |
| Pad size | 0.5 | cm² | 0.5 | cm² | 0.5 | cm² | 1 | cm² | 1 | cm² | 1 | cm² |
| (Transdermal Area) | | | | | | | | | | | | |

TABLE 29

| | AALC0022R | | |
|---|---|---|---|
| Ingredients | | G3 | G4 |

| | | | | |
|---|---|---|---|---|
| p3-Alcβ9-19 | 5 | mg | 10 | mg |
| Lactose Monohydrate | 1 | mg | 2 | mg |
| Disodium Citrate | 4.5 | mg | 4.5 | mg |
| Sesquihydrate | | | | |
| Methylparaben | 0.1 | mg | 0.1 | mg |
| Propylparaben | 0.01 | mg | 0.01 | mg |
| Pad size | 0.5 | cm² | 0.5 | cm² |
| (Transdermal Area) | | | | |

Table 30 shows PK parameters on sustained delivery with different p3-Alcβ9-19 doses using transdermal microporation in rats (100 density, 3 mJ/filament).

TABLE 30

| | AUC | | aBA* | | Cmax | | Tmax | |
|---|---|---|---|---|---|---|---|---|
| Group** | (ng/ml*hr ± SE) | | (% ± SE) | | (ng/mL ± SE) | | (hr ± SE) | |
| AALC0023R-G1 | 89.1 | 16.7 | 357.7 | 66.9 | 13.0 | 1.3 | 2.75 | 2.06 |
| AALC0023R-G2 | 157.4 | 38.9 | 379.3 | 93.8 | 26.9 | 9.4 | 0.88 | 0.13 |
| AALC0023R-G3 | 620.7 | 43.5 | 747.8 | 52.4 | 97.7 | 5.4 | 3.50 | 0.50 |
| AALC0023R-G4 | 383.8 | — | 231.2 | — | 43.5 | — | 1.00 | — |
| AALC0023R-G5 | 723.9 | — | 348.9 | — | 96.2 | — | 1.00 | — |
| AALC0026R-G2 | 333.8 | 16.1 | 402.1 | 19.4 | 37.0 | 2.2 | 0.80 | 0.10 |
| AALC0022R-G3 | 256.3 | 37.2 | 712.5 | 103.4 | 31.6 | 0.8 | 0.67 | 0.17 |
| AALC0022R-G4 | 472.2 | 29.5 | 656.1 | 41.0 | 65.0 | 6.9 | 0.67 | 0.17 |

*aBA (absolute bioavailability) vs IV (2 mg/body)
**Transdermal microporation: 100 density, 3 mJ/filament

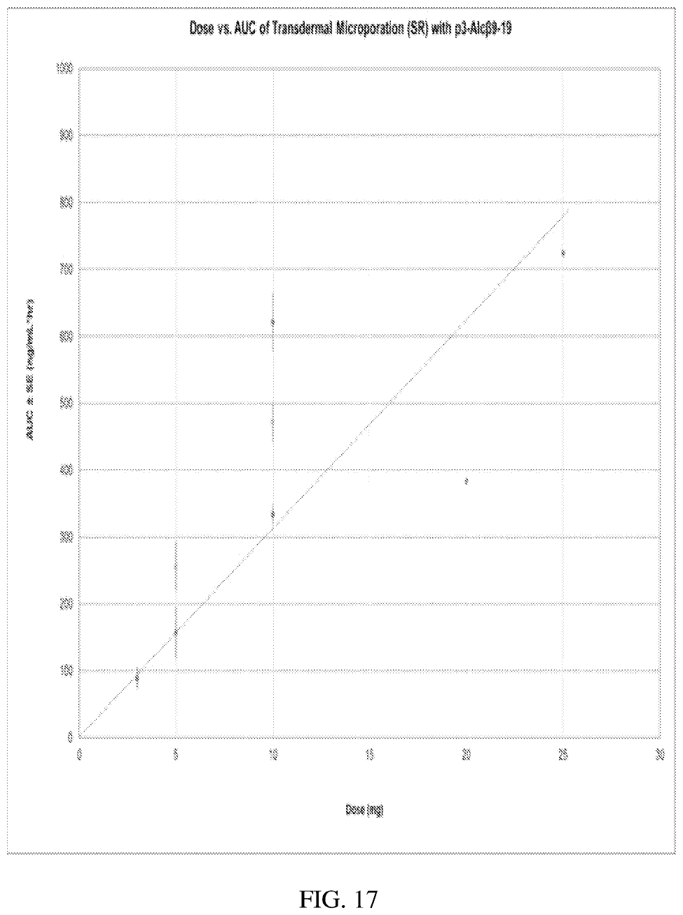

FIG. 17 illustrates the relationship between p3-Alcβ9-19 dose and AUC. The liner dose-dependency in sustained delivery was observed up to 25 mg of p3-Alcβ9-19 (tested dose).

### Example 13. Optimized Patch Formulation for p3-Alcβ Delivery

Tables 31 and 32 describes an optimized R formulations for immediate delivery and patch materials.

TABLE 31

| | | AALC0032R | | | | | |
|---|---|---|---|---|---|---|---|
| Ingredients | | G3 | | G4 | | G5 | |
| p3-Alcβ9-19 | Active Ingredient | 5 | mg | 20 | mg | 50 | mg |
| Lactose Monohydrate | Enhancer | 1 | mg | 4 | mg | 10 | mg |
| Methylparaben | Anti-Microbial Agent | 0.12 | mg | 0.48 | mg | 1.2 | mg |
| Propylparaben | Anti-Microbial Agent | 0.012 | mg | 0.048 | mg | 0.12 | mg |
| Pad size | — | 0.5 | cm² | 1 | cm² | 2 | cm² |
| (Transdermal Area) | | | | | | | |

TABLE 32

| Patch | Component | Material |
|---|---|---|
| Top Layer | Backing | Polyethylene film with acrylic adhesive |
| Middle Layer | Matrix Support | A laminated polyethylene film and |
| | | polyethylene terephthalate fiber |
| | Composition | Formulations |
| Bottom Layer | Release Liner | Silicone coated polyethylene |
| | | terephthalate film |

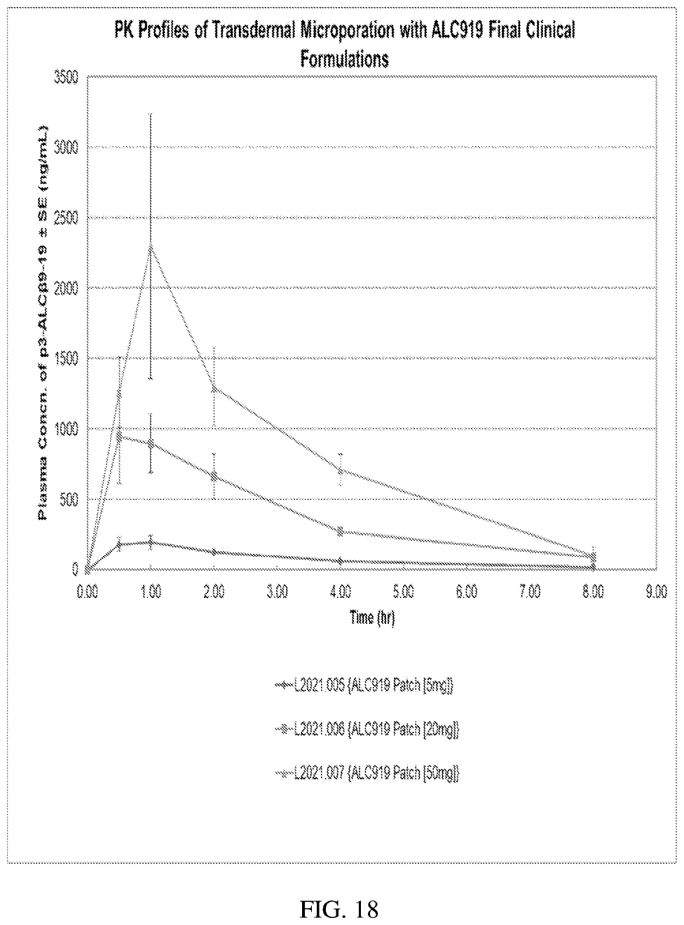

FIG. 18 illustrates a line graph depicting PK profiles of optimized formulations containing p3-Alcβ9-19 for immediate delivery in rats. Table 33 describes the results of FIG. 18. p3-Alcβ9-19 was efficiently delivered from the patch and obtained about 2000% of aBA versus IV injections. Tmax was about 1 hour.

TABLE 33

| | AUC | | aBA* | | Cmax | | Tmax | |
|---|---|---|---|---|---|---|---|---|
| Group | (ng/ml*hr ± SE) | | (% ± SE) | | (ng/mL ± SE) | | (hr ± SE) | |
| Gr. 1 | 645.3 | 99.6 | 1801.3 | 277.9 | 204.6 | 49.6 | 1.1 | 0.3 |
| Gr. 2 | 3127.0 | 600.5 | 2182.2 | 419.0 | 1033.1 | 319.8 | 0.9 | 0.1 |
| Gr. 3 | 6633.9 | 1487.4 | 1851.8 | 415.2 | 2294.4 | 936.2 | 1.0 | 0.0 |

*aBA (absolute bioavailability) vs IV (2 mg/body)
**Transdermal microporation: 400 density, 4 mJ/filament

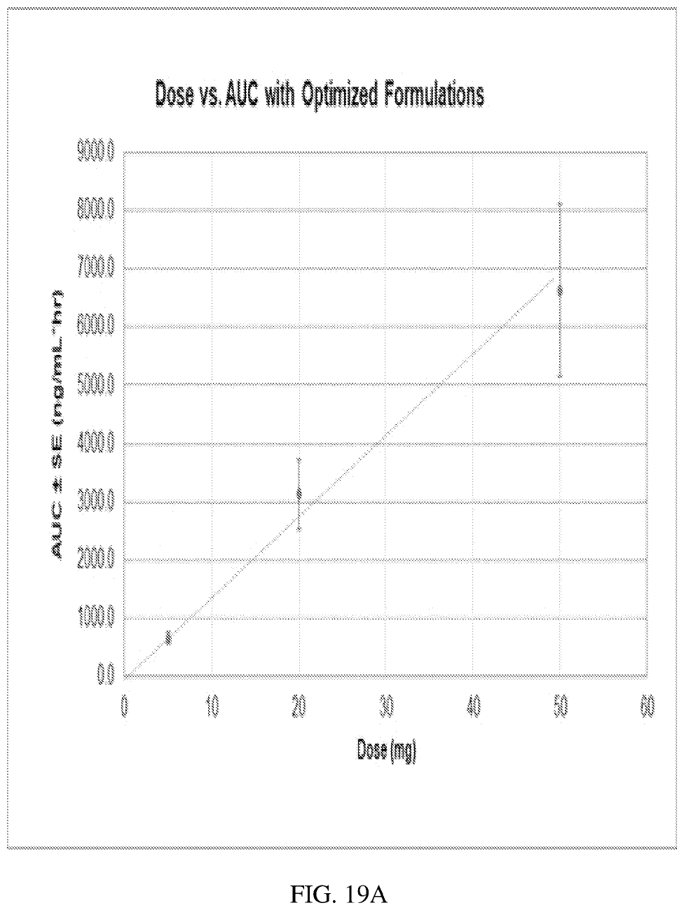

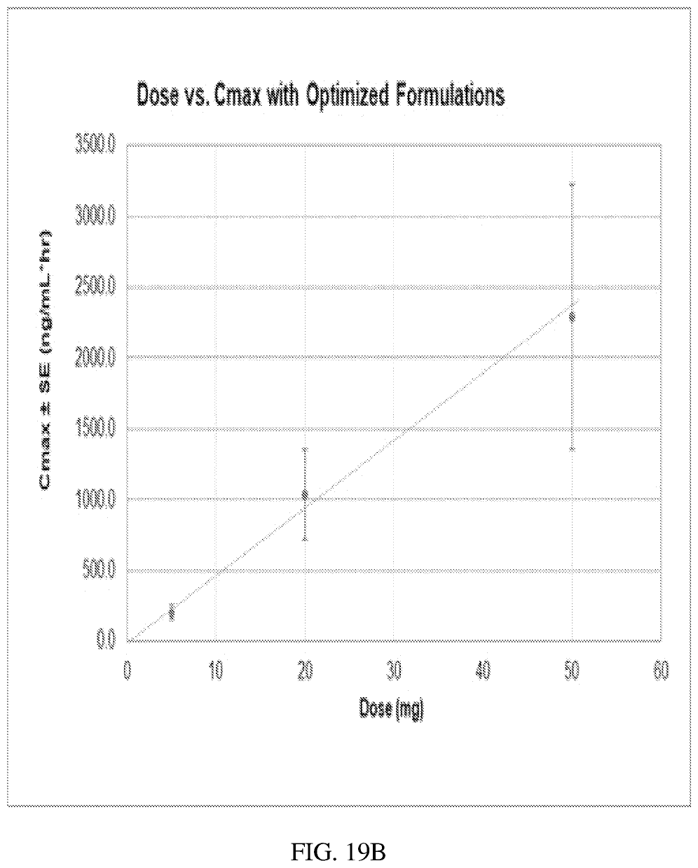

FIG. 19A describes a relationship between p3-Alcβ9-19 dose and AUC. FIG. 19B describes a relationship between p3-Alcβ9-19 dose and Cmax. AUC and Cmax were increased p3-Alcβ9-19 dose-dependently.

Physicochemical Properties of p3-Alcβ9-19 Patch. The pH and water content of the formulations were tested. Tables 34 to 36 describe the results. The pH was measured after mixing a patch in water (USP, pH 5-7). The pH of the formulations was about 4.9 (an acceptable range of pH was set between 4 and 9. The water content in a patch was measured by a volumetric Karl Fisher instrument after mixing a patch in dry methanol. The initial water content was about less than 2%. The dissolution was tested using Apparatus 5 (Paddle and disc). The patch was placed in 500 mL of phosphate buffered saline (USP) at 50 rpm, 32° C. The drug was measured by RP-HPLC. The drug was immediately released within 5-10 minutes.

TABLE 34

Test Items

| | p3-Alcβ9-19 | p3-Alcβ9-19 | p3-Alcβ9-19 |
|---|---|---|---|
| | 5 mg Patch | 20 mg Patch | 50 mg Patch |
| Test-1: pH | 4.97 ± 0.01 | 4.95 (n = 1) | 4.95 (n = 1) |
| | (n = 3) | | |
| Test-2: pH | 4.94 ± 0.03 | 4.94 ± 0.03 | 4.92 ± 0.00 |
| | (n = 3) | (n = 3) | (n = 3) |

TABLE 35

| | Test Items | | |
|---|---|---|---|
| | p3-Alcβ9-19 | p3-Alcβ9-19 | p3-Alcβ9-19 |
| | 5 mg Patch | 20 mg Patch | 50 mg Patch |
| Test-1: Water Content | 1.15 (n = 1) | — | 0.81 (n = 1) |
| (% of total solid amount) | | | |
| Test-2: Water Content | 0.77 ± 0.17 | 0.53 ± 0.30 | 0.52 ± 0.36 |
| (% of total solid amount) | (n = 3 | (n = 3) | (n = 3) |

TABLE 36

| | Test Items | | |
|---|---|---|---|
| | p3-Alcβ9-19 | p3-Alcβ9-19 | p3-Alcβ9-19 |
| | 5 mg Patch | 20 mg Patch | 50 mg Patch |
| Dissolution | Almost 100% | Almost 100% | Almost 100% |
| | released within | released within | released within |
| | 5-10 minutes | 5-10 minutes | 5-10 minutes |

Preliminary Stability of p3-Alcβ9-19 Patch. The preliminary stability of the formulations was evaluated for 3 months at 25° C./60% RH and 40° C./75% RH. Tables 37 to 39 describe the results of p3-Alcβ9-19 (5 mg) Patch, p3-Alcβ9-19 (20 mg) Patch and p3-Alcβ9-19 (50 mg) Patch, respectively. All formulations were stable at tested conditions.

TABLE 37

Test Items

| | | 3 M | 3 M |
|---|---|---|---|
| | | (25° C./ | (40° C./ |
| | Initial | 60% RH) | 75% RH) |
| Assay (p3-Alcβ9-19) | 101.7% | 104.1% | 102.5% |
| Impurity | 0.82% | 0.88% | 1.18% |
| Dissolution (released at | 103.5% | 110.0% | 112.8% |
| 30 minutes) | | | |
| Methylparaben Content | 104.1% | 99.9% | 96.1% |
| Propylparaben Content | 103.8% | 101.7% | 97.9% |
| pH | 4.94 | 4.91 | 4.91 |
| Water Content | 0.053 mg | 0.076 mg | 0.078 mg |
| (% of total solid) | (0.77%) | (1.11%) | (1.14%) |

TABLE 38

Test Items

| | | 3 M | 3 M |
|---|---|---|---|
| | | (25° C./ | (40° C./ |
| | Initial | 60% RH) | 75% RH) |
| Assay (p3-Alcβ9-19) | 99.4.% | 105.8% | 104.9% |
| Impurity | 0.72% | 0.82% | 1.02% |
| Dissolution (released at | 108.5% | 107.5% | 107.7% |
| 30 minutes) | | | |
| Methylparaben Content | 101.4% | 97.7% | 98.1% |
| Propylparaben Content | 103.4% | 101.9% | 100.8% |
| pH | 4.94 | 4.91 | 4.91 |
| Water Content | 0.145 mg | 0.683 mg | 0.390 mg |
| | (0.53%) | (2.49%) | (1.43%) |

TABLE 39

Test Items

| | | 3 M | 3 M |
|---|---|---|---|
| | | (25° C./ | (40° C./ |
| | Initial | 60% RH) | 75% RH) |
| Assay (p3-Alcβ9-19) | 98.4% | 103.5% | 103.8% |
| Impurity | 0.75% | 0.80% | 0.99% |
| Dissolution (released at | 104.6% | 105.4% | 103.3% |
| 30 minutes) | | | |
| Methylparaben Content | 99.9% | 96.8% | 95.9% |
| Propylparaben Content | 98.9% | 99.2% | 98.7% |
| pH | 4.92 | 4.91 | 4.89 |
| Water Content | 0.355 mg | 0.263 mg | 0.143 mg |
| | (0.52%) | (0.38%) | (0.21%) |

Table 40 summarizes the findings of p3-Alcβ3 administration routes. Transdermal microporation for the delivery of p3-Alcβ9-19 is an ideal administration device, and the patch formulations show good stability for room temperature storage. It is important for patient compliance and the supply chain.

TABLE 40

| Administration | PK Profile | Bioavailability (%)* |
|---|---|---|
| Oral | No absorption | 0 |
| Nasal | No absorption | 0 |
| Passive Transdermal | No absorption | 0 |
| Intravenous injection | Bolus absorption | 100 |
| Subcutaneous injection | Bolus absorption | 120-275 |
| Transdermal Microporation | Immediate or | −4800 |
| | sustained absorption | |

*Compared to IV (2 mg)

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A patch for delivering a non-aggregating peptide into a subject, the patch comprising:
a backing;
a matrix comprising a non-aggregating peptide disposed within the matrix; and
a release liner,
wherein the release liner is configured to be removed before application to a subject's skin.

2. The patch of claim 1, wherein the non-aggregating peptide is p3-Alcβ.

3. The patch of claim 2, wherein the p3-Alcβ is selected from at least one of p3-Alcβ1-40, p3-Alcβ1-37, p3-Alcβ9-19, p3-Alcβ1-19, p3-Alcβ11-19 or a derivative thereof.

4. The patch of claim 2, wherein the p3-Alcβ is p3-Alcβ9-19 or a derivative thereof.

5. The patch of any one of claims 1 to 4, wherein the non-aggregating peptide is in the matrix in an amount in the range from about 0.01 mg/cm² to about 200 mg/cm².

6. The patch of any one of claims 1 to 5, wherein the matrix further comprises at least one sugar.

7. The patch of claim 6, wherein the at least one sugar is a non-reducing sugar, a reducing sugar, or a combination thereof.

8. The patch of claim 7, wherein the non-reducing sugar is sucrose, trehalose, mannitol, sorbitol, or a combination thereof.

9. The patch of claim 7, wherein the reducing sugar is lactose, maltose, or a combination thereof.

10. The patch of any one of claims 1 to 6, wherein the weight ratio of the at least one sugar to the non-aggregating peptide is greater than about 0.02.

11. The patch of any one of claims 1 to 6, wherein the weight ratio of the at least one sugar to the non-aggregating peptide is from about 0.02 to about 0.4.

12. The patch of any one of claims 1 to 11, wherein the matrix further comprises at least one drug delivery modifier.

13. The patch of claim 12, wherein the at least one drug deliver modifier is an organic acid, a salt thereof, or a combination thereof.

14. The patch of claim 13, wherein the at least one drug delivery modifier is citric acid, its salt form, or a combination thereof.

15. The patch of any one of claims 1 to 14, wherein the matrix further comprises a preservative.

16. The patch of claim 15, wherein the preservative is an anti-microbial agent.

17. The patch of claim 16, wherein the anti-microbial agent is selected from the group consisting of methyl paraben, propylparaben, benzalkonium chloride, sodium benzoate, and combinations thereof.

18. The patch of any one of claims 1 to 17, wherein the matrix further comprises at least one of sucrose, lactose, disodium citrate sesquihydrate, methylparaben, propylparaben, and benzalkonium chloride.

19. The patch of any one of claims 1 to 18, wherein the matrix comprises at least one fiber, a laminated material of film, or a combination thereof.

20. The patch of claim 19, wherein the matrix comprises a laminated material of film and at least one fiber.

21. The patch of claim 19 or 20, wherein the at least one fiber is a non-woven fiber.

22. The patch of any one of claims 19 to 21, wherein the at least one fiber has a thickness of less than about 300 μm.

23. The patch of any one of claims 19 to 22, wherein the at least one fiber has a weight of less than about 100 g/m².

24. The patch of any one of claims 1 to 23, wherein the matrix has a water-holding capacity that is less than about 10 mg/cm².

25. A device for delivering a non-aggregating peptide into a subject through plural micropathways, a device comprising:
a patch according to any one of claim 1 to 24;
a porator comprising an array of conductive filaments; and
an applicator electrically connected to the conductive filaments and configured to supply a predetermined electrical energy to the array of conductive filaments for creating a plurality of micropores in an micropore area of a subject's skin by heating the filaments.

26. The device of claim 25, wherein the device creates between about 25 to about 500 micropathways/cm².

27. The device of 25 or 26, wherein the device has a poration energy from about 2 to about 6 mJ/filament, 4 mJ/filament, or 8 mJ/filament.

28. The device of any one of claims 25 to 27, wherein the porator is configured to open at least one channel in the subject's skin and has an area from about 0.25 cm² to about 4 cm².

29. The device of claim 28, wherein the porator is configured to open at least one channel in the subject's skin and has an area of about 0.1 cm², about 0.25 cm², about 0.65 cm², or about 1.0 cm².

30. The device of any one of claims 28 or 29, wherein the porator is configured to open at least one channel in the subject's skin and has an area less than about 1.0 cm², about 0.5 cm², or about 0.25 cm².

31. The device of claim 30, wherein the at least one channel is one or more micropores, wherein the porator is configured to generate the one or more micropores in about 0.5 to about 12.5% of the total poration area.

32. The device of any one of claims 25 to 31, wherein the porator is a microneedle, laser, or radio frequency porator configured to produce one or more micropores in the subject's skin.

33. The device of any one of claims 25 to 32, wherein the porator is configured to produce at least 50 pores in a subject's skin.

34. The device of any one of claims 25 to 33, wherein the porator is a microneedle, laser, or radio frequency porator.

35. The device of any one of claims 25 to 34, wherein the patch further includes a drug pellet.

36. A method of treating a disease or condition associated with a subject's brain, the method comprising:
opening at least one channel in a subject's skin; and
applying the patch of any one of claims 1 to 24 to the subject's skin.

37. The method of claim 36, wherein the disease or condition associated with the subject's brain is a neurodegenerative disease.

38. The method of claim 37, wherein the neurodegenerative disease is Alzheimer's disease.

39. The method of any one of claims 36 to 38, wherein opening at least one channel in the subject's skin comprises applying a transdermal microporation device to the subject's skin.

40. The method of claim 39, wherein the transdermal microporation device is a thermal tissue ablation by using a filament array having a plurality of filaments that are disposed in the skin of the subject, wherein each filament is capable of conductively delivering thermal energy via direct contact to the tissue membrane to form the plurality of micropores in a micropore area of the tissue membrane.

41. The method of claim 36 or 40, wherein the transdermal microporation apparatus creates between about 25 to about 500 micropathways/cm².

42. The method of any one of claims 39 to 41, wherein the transdermal microporation apparatus has a poration energy from about 2 to about 6 mJ/filament, about 4 mJ/filament, or about 8 mJ/filament.

43. The method of any one of claims 36 to 42, wherein opening at least one channel in the subject's skin has an area from about 0.25 cm² to about 4 cm².

44. The method of any one of claims 36 to 38, wherein the transdermal microporation apparatus is a microneedle, laser, or radio frequency device capable of producing one or more micropores in the subject's skin.

45. The method of any one of claims 36 to 44, wherein applying the patch increases neuronal viability in the subject.

46. The method of any one of claims 36 to 44, wherein applying the patch increases mitochondrial activity in the brain of the subject.

47. The method of any one of claims 36 to 46, wherein the patch provides a maximum blood concentration of the non-aggregating peptide at least 0.5 hour after administration.

48. The method of any one of claims 36 to 46, wherein the non-aggregating peptide is maintained for at least 6 hours after administration of the patch.

49. The method of any one of claims 36 to 46, wherein the non-aggregating peptide from the patch is maintained in the blood of the subject for at least 6 hours after administration of the patch to the subject.

50. The method of any one of claims 36 to 49, wherein the transfer rate of the non-aggregating peptide from the blood of the subject to the cerebrospinal fluid of the subject is at least 2%.

51. The method of claim 43, wherein opening at least one channel in the subject's skin has an area of about 0.1 cm², about 0.25 cm², about 0.65 cm², or about 1.0 cm².

52. A transdermal delivery system for delivering a non-aggregating peptide into a subject, the system comprising:
a substrate having an upper substrate surface and defining a poration area, the substrate comprising a filament array having a plurality of filaments that are disposed in the poration area, wherein each filament is capable of conductively delivering thermal energy via direct contact to the tissue membrane to form a plurality of micropores in a micropore area of the skin;
an applicator electrically connected to the filament array and configured to supply a predetermined electrical energy to the filaments for creating the plurality of micropores in the micropore area of the skin by heating the filaments;
a power supply circuit configured to provide electric current to the applicator; and
a patch according to any one of claims 1 to 24.

53. The system of claim 52, wherein the patch is for application to the one or more micropores.

54. The system of any one of claims 52 or 53, wherein the transdermal microporation device creates between about 25 to about 500 micropathways/cm².

55. The system of any one of claims 52 or 54, wherein the transdermal microporation device has a poration energy from about 2 to about 10 mJ/filament.

56. The system of any one of claims 52 to 55, wherein opening at least one channel in the subject's skin has an area from about 0.25 cm² to about 4 cm².

57. The system of any one of claims 52 to 56 wherein the one or more micropores is about 0.5 to about 12.5% of the total poration area.

58. The system of claim 52, wherein the transdermal microporation device is a microneedle, laser, or radio frequency device capable of producing one or more micropores in the subject's skin.

59. The system of any one of claims 52 to 58, wherein the transdermal microporation device produces at least 50 pores in a subject's skin.

60. The system of any one of claims 52 to 59, wherein the patch further comprises a drug pellet.

61. The system of claim 56, wherein the substrate is configured to open at least one channel in the subject's skin and has an area of about 0.1 cm², about 0.25 cm², about 0.65 cm², or about 1.0 cm².


SEQUENCE LISTING (6 sequences)

SEQ ID NO: 1 - protein, 57 aa, Homo sapiens
HVLSSQQFLHRGHQPPPEMAGHSLASSHRNSMIPSAATLHVVCVGFLVLMVVLGLVR

SEQ ID NO: 2 - protein, 40 aa, synthetic construct
VLSSQQFLHRGHQPPPEMAGHSLASSHRNSMIPSAATLHV

SEQ ID NO: 3 - protein, 37 aa, synthetic construct
VLSSQQFLHRGHQPPPEMAGHSLASSHRNSMIPSAAT

SEQ ID NO: 4 - protein, 19 aa, synthetic construct
VLSSQQFLHRGHQPPPEMA

SEQ ID NO: 5 - protein, 11 aa, synthetic construct
HRGHQPPPEMA

SEQ ID NO: 6 - protein, 9 aa, synthetic construct
GHQPPPEMA